(19) United States
(12) Patent Application Publication
(10) Pub. No.: US 2009/0264427 A1
(43) Pub. Date: Oct. 22, 2009

(54) 3-Cyanoquinolines, Methods for Preparation and Use as Insulin-like Growth Factor Inhibitors

(75) Inventors: Scott Christian Mayer, Bridgewater, NJ (US); Lori Michelle Miller, Bordentown, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/423,096

(22) Filed: Apr. 14, 2009

Related U.S. Application Data
(60) Provisional application No. 61/045,429, filed on Apr. 16, 2008.

(51) Int. Cl. A61K 31/4709; C07D 401/12; C07D 413/14; C07D 401/14; A61K 31/5377; A61K 31/496; A61P 29/00; A61P 35/00

(52) U.S. Cl. 514/235.2; 546/160; 544/128; 544/363; 514/313; 514/253.06

(57) ABSTRACT

Imidazole-substituted 4-anilino-3-cyanoquinolines are described, which selectively inhibit IFGR kinase activity and are useful for treating disorders associated with IGFR kinases.

### CROSS-REFERENCE TO RELATED APPLICATION

This application claims the benefit under 35 U.S.C. §119(e) to U.S. Provisional Application Ser. No. 61/045,429, filed Apr. 16, 2008, which is hereby incorporated by reference in its entirety.

### FIELD OF THE INVENTION

The present invention relates to new 3-cyanoquinoline compositions that are useful for inhibiting abnormal growth of certain cell types. The invention is directed to certain imidazole substituted 4-anilino-3-cyanoquinolines, their corresponding pharmaceutically acceptable salts and methods for their preparation and use as inhibitors of insulin-like growth factor receptors (IGFR). The imidazole substituted 4-anilino-3-cyanoquinolines inhibit growth of tumor cells, which contain oncogenic forms of Receptor Tyrosine Kinases and IGFR kinases.

### BACKGROUND OF THE INVENTION

Tyrosine kinases (TKs) are divided into two classes: the non-transmembrane TKs and transmembrane growth factor receptor TKs (RTKs) as described by Blume-Jensen, P., Nature, 411, 355 (2001). Growth factors, such as epidermal growth factor (EGF), bind to the extracellular domain of their partner RTK on the cell surface, which activates the RTK, initiating a signal transduction cascade that controls a wide variety of cellular responses including proliferation and migration. The overexpression of EGF and also of members of the epidermal growth factor receptor (EGFr) family, which includes EGF-r, erbB-2, erbB-3 and erbB-4, is implicated in the development and progression of cancer, as described by Rusch, V., Cytokine Growth Factor Rev., 7, 133 (1996), Davies, D. E., Biochem. Pharmacol., 51, 1101 (1996) and Modjtahedi, E., Int. J. Oncol., 4, 277 (1994). Specifically, over expression of the receptor kinase product of the erbB-2 oncogene has been associated with human breast and ovarian cancers, as described by Slamon, D. J., Science, 244, 707 (1989) and Slamon, D. J., Science, 235, 177 (1987). Upregulation of EGFr kinase activity has been associated with epidermoid tumors, as described by Reiss, M., Cancer Res., 51, 6254 (1991)]; breast tumors, as described by Macias, A., Anticancer Res., 7, 459 (1987); and tumors involving other major organs, as described by Gullick, W. J., Brit. Med. Bull., 47, 87 (1991).

In addition to EGFr, there are several other RTKs including FGFr, the receptor for fibroblast growth factor (FGF); flk-1, also known as KDR, and fit-1, the receptors for vascular endothelial growth factor (VEGF); and PDGFr, the receptor for platelet derived growth factor (PDGF). The formation of new blood vessels, a process known as angiogenesis, is essential for tumor growth. Two natural angiogenesis inhibitors, angiostatin and endostatin, dramatically inhibited the growth of a variety of solid tumors, as described by O'Reilly, M. S., Cell, 79, 315 (1994); O'Reilly, M. S., Nature Medicine, 2, 689 (1996); and O'Reilly, M. S., Cell, 88, 277 (1997). Since FGF and VEGF are known to stimulate angiogenesis, inhibition of the kinase activity of their receptors should block the angiogenic effects of these growth factors. In addition, the receptor tyrosine kinases tie-1 and tie-2 also play a key role in angiogenesis, as described by Sato, T. N., Nature, 376, 70 (1995). Compounds of that inhibit the kinase activity of FGFr, flk-1, fit-1, tie-1 or tie-2 may inhibit tumor growth by their effect on angiogenesis.

The type 1 insulin-like growth factor receptor (IGF1R) is a transmembrane receptor tyrosine kinase that binds primarily to IGF1 but also to IGF2 and insulin with lower affinity. Binding of IGF1 to its receptor results in receptor oligomerization, activation of tyrosine kinase, intermolecular receptor autophosphorylation and phosphorylation of cellular substrates (its two major substrates are IRS1 and Shc). The ligand-activated IGF1R induces mitogenic activity in normal cells. Several clinical reports underline the important role of the IGF-I pathway in human tumor development: (i) IGF-I-R over-expression is frequently found in various tumors (for example breast, colon, lung, skin, sarcoma) and is often associated with an aggressive phenotype; (ii) high circulating IGF1 concentrations are strongly correlated with prostate, lung and breast cancer risk; (iii) epidemiological studies implicate the IGF1 axis as a predisposing factor in the pathogenesis of breast and prostate cancer, as described by Baserga R., “The IGF-I receptor in cancer research,” Exp Cell Res. (1999) 253:1-6; Baserga R., “The contradictions of the IGF1-Receptor,” Oncogene (2000) 19: 5574-81; Khandwala H M. et al., “The effects of IGFs on tumorigenesis and neoplastic growth,” Endocrine Reviews (2000) 21: 215-44; and Adams T E et al., “Structure and function of the IGF1R,” CMLS (2000) 57: 1050-93.

U.S. Pat. No. 6,698,772 describes certain 3-cyanoquinolines as inhibitors of a certain protein tyrosine kinase and the treating of polycystic kidney diseases associated with the tyrosine kinase. The patent does not disclose imidazole groups substituted at the phenyl ring of 4-anilino-3-cyanoquinoline compounds. The present invention more completely discloses how functionalized imidazole groups substituted at the phenyl ring of the 4-anilino-3-cyanoquinoline ring framework influence structure-activity relationships (SAR) of 4-anilino-3-cyanoquinoline compounds. There is a need for new compounds that selectively inhibit IGFR kinase activity and that are useful for treating disorders associated with by any IGFR kinase. Imidazole substituted, 4-anilino-3-cyanoquinoline compounds of the present invention fulfill this unmet need and are useful in the treatment of specific diseases associated with certain IGFR kinases in mammals.

### SUMMARY OF THE INVENTION

Accordingly, the invention provides a compound of formula I:

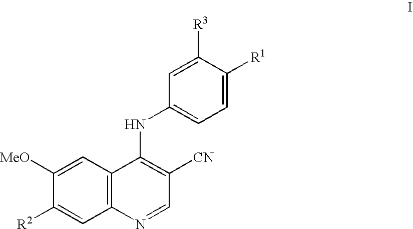

and pharmaceutically acceptable salts thereof;
wherein

### R¹ is

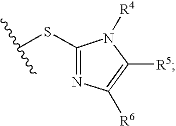

### R² is —XCH₂CH₂CH₂Y;

R³ is selected from H, F, Cl, Br and I;
R⁴, R⁵ and R⁶ are each independently selected from unbranched alkyl of 1-6 carbon atoms, branched alkyl of 3-8 carbon atoms, aryl and benzyl, or R⁵ and R⁶ join together to form a fused 6-membered aryl ring comprising CR⁷ or heteroaryl ring comprising CR⁷ and N;
R⁷ and R³ are each independently selected from H, unbranched alkyl of 1-6 carbon atoms, branched alkyl of 3-8 carbon atoms, hydroxyalkyl of 1-6 carbon atoms, dihydroxyalkyl of 1-6 carbon atoms, alkylsulfonyl of 1-6 carbon atoms, aryl sulfonyl, and —CH₂CH₂-heterocycle;

### X is —O—, —N(H)—, or —N(Me)—; and

Y is selected from NR⁷R⁸, aryl, and a 5- to 6-membered heterocycle comprising 1-2 heteroatoms selected from N and O, said aryl and heterocycle substituted with 0-3 substituents selected from unbranched alkyl of 1-6 carbon atoms, branched alkyl of 3-8 carbon atoms, hydroxyalkyl of 1-6 carbon atoms, —OH, acetyl, —CO₂-alkyl of 1-6 carbon atoms, and —CH₂CO₂-alkyl of 1-6 carbon atoms.

The present invention also provides a pharmaceutical composition comprising a compound of formula I and a pharmaceutically acceptable carrier. The present invention also provides a pharmaceutical composition comprising a compound of formula I in combination with one or more kinase-inhibiting compound or chemotherapeutic agent and a pharmaceutically acceptable carrier.

The present invention provides a method for making a compound of formula I comprising the step of:

reacting a compound of formula 1:

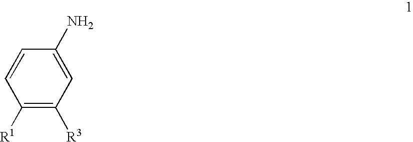

with a compound of formula 2:

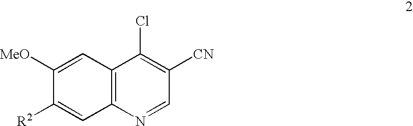

wherein R¹-R³ are defined above.

The present invention also provides a method of treating an IGFR kinase dependent condition, said condition comprising cancer or inflammation, by administering to a patient a pharmaceutically effective amount of a compound of formula I.

The present invention provides a method of treating mammalian diseases associated with an IGFR kinase by administering, to a patient, a compound of formula I.

The present invention provides a method of treating cancer by administering, to a patient, a compound of formula I, wherein the cancer is selected from the group consisting of: breast, kidney, bladder, thyroid, mouth, larynx, esophagus, stomach, colon, ovary, lung, pancreas, skin, liver, prostate and brain cancer.

### DETAILED DESCRIPTION OF THE INVENTION

### Definitions

The term “alkyl” refers to the radical of saturated aliphatic groups of 1 to 8 carbon atoms, including unbranched-chain alkyl groups, branched-chain alkyl groups, cycloalkyl (alicyclic) groups, alkyl substituted cycloalkyl groups, and cycloalkyl substituted alkyl groups. In one embodiment, an unbranched chain alkyl has 6 or fewer carbon atoms in its backbone and a branched chain alkyl has 3 to 8 carbon atoms in its backbone. The term “alkyl” can be used alone or as part of a chemical name, such as “alkylamine”. The terms “alkenyl” and “alkynyl” refer to unsaturated aliphatic groups analogous in length and possible substitution to the alkyls described above, but which contain at least one double or triple carbon-carbon bond, respectively.

The term “aryl”, as used herein, whether used alone or as part of another group, is defined as a substituted or unsubstituted aromatic hydrocarbon ring group having 6 to about 50 carbon atoms (unless explicitly specified otherwise) with from about 6 to about 10 atoms being preferred. The “aryl” group can have a single ring or multiple condensed rings. The term “aryl” includes, but is not limited to phenyl, α-naphthyl, β-naphthyl, biphenyl, anthryl, tetrahydronaphthyl, fluorenyl, indanyl, biphenylenyl, and acenaphthenyl. Specifically included within the definition of “aryl” are those aromatic groups that are optionally substituted. For example, in representative embodiments of the present invention, the, “aryl” groups are optionally substituted with from 1 to 4 substituents selected from: H, C₁-C₃ alkyl, F, Cl, Br, I, CF₃, NO₂, —NR⁷R¹, —CHO, —CONHPh, —CO₂R⁷, —SO₂R⁷, and —SO₂R⁷, where R⁷ and R³ are defined above.

The term “heteroaryl” as used herein is defined as a substituted or unsubstituted aromatic heterocyclic ring system (monocyclic or bicyclic). Heteroaryl groups can have, for example, from about 3 to about 50 carbon atoms and 1-8 heteroatoms (unless explicitly specified otherwise) with from about 4 to about 10 carbon atoms and 1-4 heteroatoms being preferred. In some embodiments, heteroaryl groups are aromatic heterocyclic rings systems having about 4 to about 14 ring atoms including carbon atoms and 1, 2, 3, or 4 heteroatoms selected from oxygen, nitrogen or sulfur. Bicyclic aromatic heteroaryl groups include phenyl, pyridine, pyrimidine or pyridizine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Specifically included within the definition of “heteroaryl” are those aromatic groups that are optionally substituted. Accordingly, the heteroaryl groups described herein include both unsubstituted or substituted groups. Suitable examples of monocyclic and bicyclic heteroaryl groups are selected from: furan, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, benzoxazole, benzothiazole, benzofuran, benzisoxazole, benzimidazole, N-methylbenzimidazole, azabenzimidazole, indazole, quinazoline, quinoline, and isoquinoline. In representative embodiments of the present invention, the, “heteroaryl” groups are optionally substituted with 1 to 4 substituents selected from: H, C₁-C₃ alkyl, F, Cl, Br, I, CF₃, NO₂, —NR⁷R⁸, —CHO, —CONHPh, —CO₂R⁷, —SO₂R⁷, and —SO₂R⁷, where R⁷ and R³ are defined above.

The term “heterocycle”, as used herein, whether used alone or as part of another group, refers to a stable 3 to about 10-member ring containing carbons atoms and from 1 to 4 heteroatoms selected from the group consisting of nitrogen, oxygen, and sulfur. A heterocycle of this invention can be either a monocyclic or bicyclic ring system, and can be either saturated, unsaturated, or partially saturated. A heterocycle can be optionally fused to a phenyl ring or a thiazole ring. The term “heterobicyclic ring” refers to a bicyclic ring system comprising at least one heterocycle. Suitable examples of heterocycles include, but are not limited to, aziridinyl, azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothienyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dihydro-1,4-dioxanyl, tetrahydrofuranyl, tetrahydrothienyl, tetrahydroquinolinyl, and tetrahydroisoquinolinyl. Preferred heterocycle moieties include: (a) 6-membered saturated, partially unsaturated, or unsaturated heterocycles containing 1-2 nitrogens, optionally fused to a phenyl ring; (b) 5-membered saturated, partially saturated, or unsaturated heterocycles containing 1-3 nitrogen, oxygen, or sulfur atoms, optionally fused to a phenyl ring; (c) saturated, partially unsaturated, or unsaturated bicyclic heterocycles containing 1-4 nitrogen, oxygen, or sulfur atoms; (d) carbazole, dibenzofuran, and dibenzothiophene. Specifically included in the definition of “heterocycle” are those heterocycles that are optionally substituted with one to four substituents selected from: H, C₁-C₃ alkyl, F, Cl, Br, I, CF₃, NO₂, —NR⁷R⁸, —CHO, —CONHPh, —CO₂R⁷, —SO₂R⁷, and —SO₂R⁷, where R⁷ and R⁸ are defined above.

The term “halogen” refers to an atom of fluorine, chlorine, bromine, or iodine. As used herein, the term “substituted” is contemplated to include all permissible substituents of organic compounds. Typically, suitable substituents of organic compounds include acyclic and cyclic, branched and unbranched, carbocyclic and heterocyclic, aromatic and nonaromatic substituents of organic compounds as well as inorganic substituents such as halogen or amino. The substituents can be one or more and the same or different for appropriate organic compounds. For purposes of this invention, the heteroatoms such as nitrogen may have hydrogen substituents, halogen substituents and/or any suitable substituents of organic compounds described herein which satisfy the valencies of the heteroatoms. This invention is not intended to be limited in any manner by the suitable substituents of organic compounds.

As used herein, the term “pharmaceutically acceptable carrier” includes pharmaceutically acceptable diluents and excipients.

As used herein, the term “individual”, “subject” or “patient,” used interchangeably, refers to any animal, including mammals, preferably mice, rats, other rodents, rabbits, dogs, cats, swine, cattle, sheep, horses, or primates, and most preferably humans.

Accordingly, the invention provides a compound of formula I:

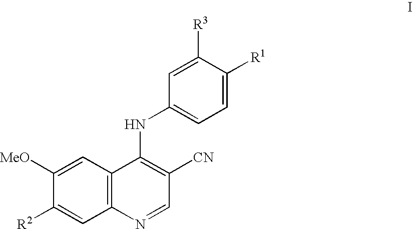

and pharmaceutically acceptable salts thereof;
wherein

### R¹ is

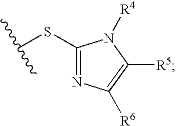

### R² is —XCH₂CH₂CH₂Y;

R³ is selected from H, F, Cl, Br and I;
R⁴, R⁵ and R⁶ are each independently selected from unbranched alkyl of 1-6 carbon atoms, branched alkyl of 3-8 carbon atoms, aryl and benzyl, or R⁵ and R⁶ join together to form a fused 6-membered aryl ring comprising CR⁷ or heteroaryl ring comprising CR⁷ and N;
R⁷ and R³ are each independently selected from H, unbranched alkyl of 1-6 carbon atoms, branched alkyl of 3-8 carbon atoms, hydroxyalkyl of 1-6 carbon atoms, dihydroxyalkyl of 1-6 carbon atoms, alkylsulfonyl of 1-6 carbon atoms, aryl sulfonyl, and —CH₂CH₂-heterocycle;

### X is —O—, —N(H)—, or —N(Me)—; and

Y is selected from NR⁷R³, aryl, and a 5- to 6-membered heterocycle comprising 1-2 heteroatoms selected from N and O, said aryl and heterocycle substituted with 0-3 substituents selected from unbranched alkyl of 1-6 carbon atoms, branched alkyl of 3-8 carbon atoms, hydroxyalkyl of 1-6 carbon atoms, —OH, acetyl, —CO₂-alkyl of 1-6 carbon atoms, and —CH₂CO₂-alkyl of 1-6 carbon atoms.

Compounds of the invention inhibit IGFR kinase activity and inhibition increases with increasing substitution of functional groups at the imidazole ring of the 4-substituted aniline. According to one embodiment, R¹ is an imidazole ring substituted with 1-3 substituents of formula:

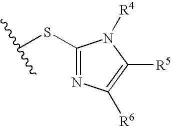

wherein all of R⁴, R⁵ and R⁶ are each independently selected from alkyl of 1-6 carbon atoms, aryl and benzyl. According to a separate embodiment, R¹ is at least a di-substituted imidazole ring, wherein R⁴, R⁵ and R⁶ are each independently selected from alkyl of 1-6 carbon atoms, aryl and benzyl.

According to another embodiment, R¹ is at least a tri-substituted imidazole ring, wherein R⁴, R⁵ and R⁵ are each independently selected from alkyl of 1-6 carbon atoms, aryl and benzyl, or R⁵ and R⁶ join together to form a fused 6-membered aryl ring comprising CR⁷ or heteroaryl ring comprising CR⁷ and N. In one preferred embodiment, R⁵ and R⁶ are each methyl and R⁴ is selected from methyl, ethyl and isopropyl. In another embodiment, R⁵ and R⁶ join together to form an unsubstituted benzoimidazole ring or a substituted benzoimidazole ring, wherein R⁷ is defined above and R⁴ is selected from methyl, ethyl and isopropyl.

In some embodiments, R² is —XCH₂CH₂CH₂Y, where X is —N(H)— or —N(Me)— and Y is NR⁷R⁸, phenyl or a 5- to 6-membered heterocycle comprising 1-2 heteroatoms selected from N and O, said aryl and heterocycle substituted with 0-3 substituents. In other embodiments, R² is —XCH₂CH₂CH₂Y, where X is —O— and Y is a 6-membered heterocycle comprising 1-2 heteroatoms selected from N and O, said heterocycle substituted with 0-3 substituents.

Suitable examples of compounds of the invention include, but are not limited to compounds selected from 4-({3-Chloro-4-[(1,4,5-trimethyl-1H-imidazol-2-yl)thio]phenyl}amino-7-{[3-(dimethylamino)propyl]amino}-6-methoxyquinoline-3-carbonitrile, 4-({4-[(1-benzyl-4,5-dimethyl-1H-imidazol-2-yl)thio]-3-chloro-phenyl}amino)-7-{[3-(dimethylamino)propyl]amino}-6-methoxyquinoline-3-carbonitrile, 4-({3-chloro-4-[(1,5-dimethyl-1H-benzimidazol-2-yl)thio]phenyl}amino)-7-{[3-(dimethylamino)propyl]amino}-6-methoxyquinoline-3-carbonitrile, 4-({3-chloro-4-[(1-ethyl-4,5-dimethylamino)-propyl]amino-6-methoxyquinoline-3-carbonitrile, 4-({3-bromo-4-[(1,4,5-trimethyl-1H-imidazol-2-yl)thio]phenyl}amino)-7-{[3-(dimethylamino)-propyl]amino}-6-methoxy-quinoline-3-carbonitrile, 7-{[3-(dimethyl-amino)propyl]-amino}-6-methoxy-4-({4-[(1,4,5-trimethyl-1H imidazol-2-yl)thio]phenyl}amino)-quinoline-3-carbonitrile, 4-({3-Chloro-4-[(1-ethyl-4,5-dimethyl-1H-imidazol-2-yl)thio]phenyl}-amino)-6-methoxy-7-[(3-phenylpropyl)amino]quinoline-3-carbonitrile, 4-({3-chloro-4-[(1-ethyl-4,5-dimethyl-1H-imidazol-2-yl)thio]phenyl}-amino)-6-methoxy-7-[(3-morpholin-4-ylpropyl)amino]-quinoline-3-carbonitrile, 7-({3-[bis(2-hydroxyethyl)amino]propyl}amino)-4-({3-chloro-4-[(1-ethyl-4,5-dimethyl-1H-imidazol-2-yl)thio]phenyl}amino)-6-methoxyquinoline-3-carbonitrile, N-(3-{4-[3-chloro-4-(1-ethyl-4,5-dimethyl-1H-imidazol-2-ylsulfanyl)-phenylamino]-3-cyano-6-methoxy-quinolin-7-yloxy}-propyl)-benzenesulfonamide, 7-{3-[tert-butyl(2-hydroxy-ethyl)amino]propoxy}-4-({3-chloro-4-[(1-ethyl-4,5-dimethyl-1H-imidazol-2-yl)thio]phenyl}amino)-6-methoxyquinoline-3-carbonitrile, 4-({3-chloro-4-[(1-ethyl-4,5-dimethyl-1H-imidazol-2-yl)thio]phenyl}amino)-7-[3-(dimethyl-amino)propoxy]-6-methoxyquinoline-3-carbonitrile, ethyl 4-(3-{[4-({3-chloro-4-[(1-ethyl-4,5-dimethyl-1H-imidazol-2-yl)thio]phenyl}amino)-3-cyano-6-methoxyquinolin-7-yl]oxy}propyl)-piperazine-1-carboxylate, 4-({3-chloro-4-[(1-ethyl-4,5-dimethyl-1H-imidazol-2-yl)thio]phenyl}amino)-7-[3-(2-ethylpiperidin-1-yl)propoxy]-6-methoxy-quinoline-3-carbonitrile, ethyl 1-(3-{[4-({3-chloro-4-[(1-ethyl-4,5-dimethyl-1H-imidazol-2-yl)thio]phenyl}amino)-3-cyano-6-methoxyquinolin-7-yl]oxy}propyl)-piperidine-4-carboxylate, N-(3-{[4-({3-chloro-4-[(1-ethyl-4,5-dimethyl-1H-imidazol-2-yl)thio]phenyl]amino)-3-cyano-6-methoxyquinolin-7-yl]oxy}propyl)-methanesulfonamide, 4-({3-Chloro-4-[(1-ethyl-4,5-dimethyl-1H-imidazol-2-yl)sulfanyl]phenyl}amino)-7-[3-(4-ethyl-1-piperazinyl)-propoxy]-6-methoxy-3-quinolinecarbonitrile, 4-({3-chloro-4-[(1-ethyl-4,5-dimethyl-1H-imidazol-2-yl)thio]-phenyl}amino)-7-{3-[4-(2-hydroxyethyl)piperazin-1-yl]propoxy}-6-methoxy-quinoline-3-carbonitrile, 4-({3-chloro-4-[(1-ethyl-4,5-dimethyl-1H-imidazol-2-yl)thio]phenyl}amino)-7-{3-[(2-hydroxyethyl)(methyl)amino]propoxy}-6-methoxy-quinoline-3-carbonitrile, 4-({3-chloro-4-[(1-ethyl-4,5-dimethyl-1H-imidazol-2-yl)thio]phenyl}amino)-7-[3-(3-hydroxypyrrolidin-1-yl)propoxy]-6-methoxyquinoline-3-carbonitrile, 4-({3-chloro-4-[(1-ethyl-4,5-dimethyl-1H-imidazol-2-yl)thio]phenyl}amino)-6-methoxy-7-(3-pyrrolidin-1-ylpropoxy)qquinoline-3-carbonitrile, ethyl [4-(3-{[4-({3-chloro-4-[(1-ethyl-4,5-dimethyl-1H-imidazol-2-yl)thio]phenyl}amino)-3-cyano-6-methoxyquinolin-7-yl]oxy}propyl)piperazin-1-yl]acetate, 4-({3-chloro-4-[(1-ethyl-4,5-dimethyl-1H-imidazol-2-yl)thio]phenyl}amino)-7-{3-[(2,3-dihydroxypropyl)(methyl)-amino]propoxy}-6-methoxyquinoline-3-carbonitrile, 4-({3-chloro-4-[(1-ethyl-4,5-dimethyl-1H-imidazol-2-yl)thio]phenyl}amino)-6-methoxy-7-(3-morpholin-4-ylpropoxy)qquinoline-3-carbonitrile, 4-({3-chloro-4-[(1-ethyl-4,5-dimethyl-1H-imidazol-2-yl)thio]phenyl}amino)-7-{3-[[2-(1,3-dioxolan-2-yl)ethyl](methyl)amino]propoxy}-6-methoxyquinoline-3-carbonitrile, 4-({3-chloro-4-[(1-ethyl-4,5-dimethyl-1H-imidazol-2-yl)thio]phenyl}amino)-7-[3-(4-hydroxypiperidin-1-yl)propoxy]-6-methoxyquinoline-3-carbonitrile, 7-[3-(4-acetylpiperazin-1-yl)propoxy]-4-({3-chloro-4-[(1-ethyl-4,5-dimethyl-1H imidazol-2-yl)thio]phenyl}amino)-6-methoxyquinoline-3-carbonitrile, 4-[3-Chloro-4-(1-ethyl-4,5-dimethyl-1H-imidazol-2-ylsulfanyl)-phenylamino]-6-methoxy-7-[3-(4-methyl-piperazin-1-yl)-propylamino]-quinoline-3-carbonitrile, 4-[3-Chloro-4-(4,5-dimethyl-1-propyl-1H-imidazol-2-ylsulfanyl)-phenylamino]-7-(3-dimethylamino-propylamino)-6-methoxy-quinoline-3-carbonitrile, 4-[3-Chloro-4-(1-isopropyl-4,5-dimethyl-1H-imidazol-2-ylsulfanyl)-phenylamino]-7-(3-dimethylamino-propylamino)-6-methoxy-quinoline-3-carbonitrile and pharmaceutically acceptable salts thereof.

Where present, compounds of formula I and corresponding pharmaceutically acceptable salts or esters thereof include isomers either individually or as a mixture, such as enantiomers, diastereomers, and positional isomers. “Pharmaceutically acceptable salts and esters” refers to salts and esters that are pharmaceutically acceptable and have the desired pharmacological properties. Such salts include, for example, salts that can be formed where acidic protons present in the compounds are capable of reacting with inorganic or organic bases. Suitable inorganic salts include, for example, those formed with the alkali metals or alkaline earth metals, e.g. sodium and potassium, magnesium, calcium, and aluminum. Suitable organic salts include, for example, those formed with organic bases such as the amine bases, e.g. ethanolamine, diethanolamine, triethanolamine, tromethamine, N-methylglucamine, and the like. Pharmaceutically acceptable salts can also include acid addition salts formed from the reaction of basic moieties, such as amines, in the parent compound with inorganic acids (e.g. hydrochloric and hydrobromic acids) and organic acids (e.g. acetic acid, citric acid, maleic acid, and the alkane- and arene-sulfonic acids such as methanesulfonic acid and benzenesulfonic acid).

Pharmaceutically acceptable esters include esters formed from carboxy, sulfonyloxy, and phosphonoxy groups present in the compounds, e.g. C₁₋₆ alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention can be present in more than one stereoisomeric form, and the naming of such compounds is intended to include all single stereoisomers and all mixtures (whether racemic or otherwise) of such stereoisomers.

Pharmaceutically acceptable salts of compounds of formula I with an acidic moiety may be formed from organic and inorganic bases. For example with alkali metals or alkaline earth metals such as sodium, potassium, lithium, calcium, or magnesium or organic bases and N-tetraalkylammonium salts such as N-tetrabutylammonium salts. Similarly, when a compound of this invention contains a basic moiety, salts may be formed from organic and inorganic acids. For example salts may be formed from acids: acetic, propionic, lactic, citric, tartaric, succinic, fumaric, maleic, malonic, mandelic, malic, phthalic, hydrochloric, hydrobromic, phosphoric, nitric, sulfuric, methanesulfonic, naphthalenesulfonic, benzenesulfonic, toluenesulfonic, camphorsulfonic, and similarly known acceptable acids when a compound of this invention contains a basic functional group. Other suitable examples of pharmaceutically acceptable salts include, but are not limited, to sulfate; citrate, acetate; oxalate; chloride; bromide; iodide; nitrate; bisulfate; phosphate; acid phosphate; isonicotinate; lactate; salicylate; acid citrate; tartrate; oleate; tannate; pantothenate; bitartrate; ascorbate; succinate; maleate; gentisinate; fumarate; gluconate; glucaronate; saccharate; formate; benzoate; glutamate; methanesulfonate; ethanesulfonate; benzenesulfonate; p-toluenesulfonate; pamoate (i.e., 1,1′-methylene-bis-(2-hydroxy-3-naphthoate)); and salts of fatty acids such as caproate, laurate, myristate, palmitate, stearate, oleate, linoleate, and linolenate salts. The compounds can also be used in the form of esters, carbamates and other conventional ester forms, also refereed to herein as prodrug forms, which when administered in such form, convert to the active moiety in-vivo. Exemplary ester forms of the compounds of this invention include, but are not limited to, straight chain alkyl esters having from 1 to 6 carbon atoms or branched chain alkyl groups containing 1 to 6 carbon atoms, including methyl, ethyl, propyl, butyl, 2-methylpropyl and 1,1-dimethylethyl esters, cycloalkyl esters, alkylaryl esters, benzyl esters, and the like.

The compounds of this invention may be prepared from: (a) commercially available starting materials (b) known starting materials which may be prepared as described in literature procedures or (c) new intermediates described in the schemes and experimental procedures herein.

Reactions are performed in a solvent appropriate to the reagents and materials employed and suitable for the transformation being effected. It is understood by those skilled in the art of organic synthesis that the various functionalities present on the molecule must be consistent with the chemical transformation proposed. This may necessitate judgement as to the order of synthetic steps.

The synthetic route for preparing the imidazole substituted aniline group of formula A, is summarized in Scheme 1.

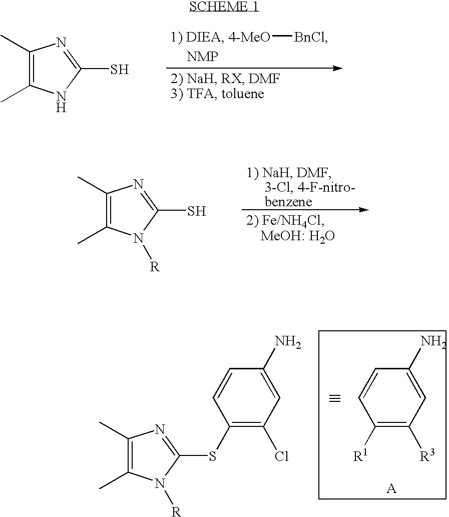

Starting from 4,5-dimethyl-1H-imidazole-2-thiol, the thiol group is protected with 4-methoxybenzylchloride. Using a reducing agent (e.g. NaH) and an alkyl halide (e.g. iodoethane) provides an N-alkylated imidazole. The 2-thiol group is then deprotected. Treating the 2-thiol with a reducing agent (e.g. NaH) followed by addition of 3-chloro-4-fluoronitrobenzene results in imidazole substituted nitrobenzene (e.g. 2-(2-chloro-4-nitro-phenylsulfanyl)-1-ethyl-4,5-dimethyl-1H-imidazole). Reduction of the nitro group with iron powder provides the desired imidazole substituted aniline group of formula A.

The synthetic route for preparing compounds of the invention where R² is a N-bonded functional group (X is NH or NMe) is summarized in Scheme 2.

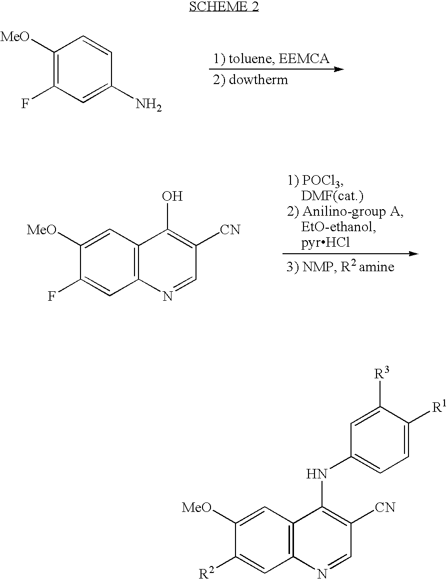

Referring to Scheme 2, the intermediate compound, 7-Fluoro-4-hydroxy-6-methoxy-quinoline-3-carbonitrile, is prepared as described in U.S. Pat. No. 6,288,082. The intermediate is chlorinated to provide the compound 7-Fluoro-4-chloro-6-methoxy-quinoline-3-carbonitrile. Addition of the imidazole substituted aniline group of formula A is accomplished using pyridine HCl. Nucleophilic displacement of fluoride using an amine (e.g. amine of the R² group) results in the desired compounds of the invention.

The synthetic route for preparing compounds of the invention where R² is a O-bonded functional group (X is O) is summarized in Scheme 3.

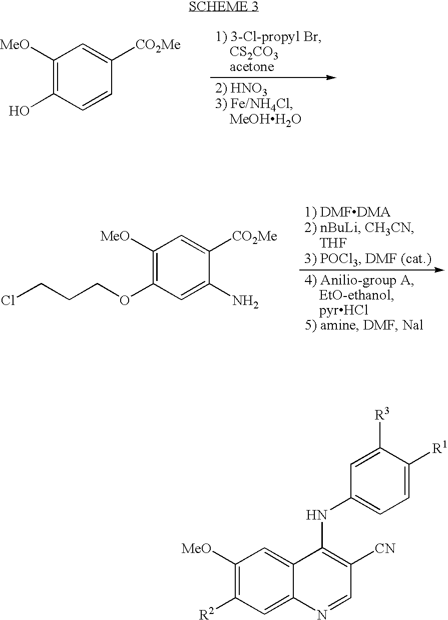

Referring to Scheme 3, methyl vanillate is functionalized with a chloropropoxy group by treating the reactant with 3-chloropropylbromide and a base (e.g. cesium carbonate) in acetone to provide methyl 4-(3-chloropropoxy)-3-methoxybenzoate. Nitration using nitric acid provides methyl 4-(3-chloropropoxy)-5-methoxy-2-nitrobenzoate. Reduction of the nitro group using iron provides methyl 2-amino-4-(3-chloropropoxy)-5-methoxybenzoate. Reaction with DMF-DMA followed by cyclization with the lithium anion of acetonitrile provides 7-(3-chloropropoxy)-4-hydroxy-6-methoxyquinoline-3-carbonitrile. Chlorination followed by addition of the imidazole substituted aniline group and substituted amines provides compounds of the invention.

The present invention accordingly provides a pharmaceutical composition, which comprises an effective amount of a compound of the present invention in combination or association with a pharmaceutically acceptable carrier. Suitable examples of pharmaceutical carriers used in accordance with the present invention include, but are not limited to, excipients, diluents, fillers, disintegrants, lubricants and other agents that can function as a carrier. The term “pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. Pharmaceutical compositions are prepared in accordance with acceptable pharmaceutical procedures, such as described in Remingtons Pharmaceutical Sciences, 17th edition, ed. Alfonoso R. Gennaro, Mack Publishing Company, Easton, Pa. (1985). Pharmaceutically acceptable carriers are those that are compatible with the other ingredients in the formulation and biologically acceptable. As used herein, the term “effective amount” refers to the amount of active compound or pharmaceutical agent that elicits the biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease; (2) inhibiting the disease; for example, inhibiting a disease, condition or disorder in an individual that is experiencing or displaying the pathology or symptomatology of the disease, condition or disorder (i.e., arresting or slowing further development of the pathology and/or symptomatology); and (3) ameliorating the disease; for example, ameliorating a disease, condition or disorder in an individual that is experiencing or displaying the pathology or symptomatology of the disease, condition or disorder (i.e., reversing the pathology and/or symptomatology).

The term “treating” or “treatment” refers to any indicia of success in amelioration of an injury, pathology, or condition, including any objective or subjective parameter such as abatement; remission; diminishing of symptoms or making the injury, pathology, or condition more tolerable to the patient; slowing the rate of degeneration or decline; making the final point of degeneration less debilitating; or improving a subject's physical or mental well-being. The treatment or amelioration of symptoms can be based on objective or subjective parameters; including the results of a physical examination, neurological examination, and/or psychiatric evaluation. “Treating” or “treatment of a securin related disorder” includes preventing the onset of symptoms in a subject that may be predisposed to a securin related disorder but does not yet experience or exhibit symptoms of the disorder (prophylactic treatment), inhibiting the symptoms of the disorder (slowing or arresting its development), providing relief from the symptoms or side-effects of the disorder (including palliative treatment), and/or relieving the symptoms of the disorder (causing regression). Accordingly, the term “treating” includes the administration of the compounds or agents of the present invention to a subject to prevent or delay, to alleviate, or to arrest or inhibit development of the symptoms or conditions associated with IGF1-R kinase related disorders, e.g., tumor growth associated with cancer. A skilled medical practitioner will know how to use standard methods to determine whether a patient is suffering from a disease associated with activity of an IGF1-R kinase, e.g., by examining the patient and determining whether the patient is suffering from a disease known to be associated with IGF1-R kinase activity or by assaying for IGF1-R kinase levels in blood plasma or tissue of the individual suspected of suffering from an IGF1-R kinase related disease and comparing securin levels in the blood plasma or tissue of the individual suspected of suffering from a IGF1-R kinase related disease to IGF1-R kinase levels in the blood plasma or tissue of a healthy individual. Increased securin levels are indicative of disease. Accordingly, the present invention provides, inter alia, methods of administering a compound of the present invention to a subject and determining IGF1-R kinase activity in the subject. IGF1-R kinase activity in the subject can be determined before and/or after administration of the compound.

The term “IGFR kinase related disorder or disease associated with IGF1-R kinase activity” refers to any disease or condition that is associated with increased or enhanced expression or activity of IGF1-R kinase or increased or enhanced expression or activity of a gene encoding IGF1-R kinase. Examples of such increased activity or expression can include one or more of the following: activity of the protein or expression of the gene encoding the protein is increased above the level of that in normal subjects; activity of the protein or expression of the gene encoding the protein is in an organ, tissue or cell where it is not normally detected in normal subjects (i.e. spatial distribution of the protein or expression of the gene encoding the protein is altered); activity of the protein or expression of the gene encoding the protein is increased when activity of the protein or expression of the gene encoding the protein is present in an organ, tissue or cell for a longer period than in a normal subjects (i.e., duration of activity of the protein or expression of the gene encoding the protein is increased). A normal or healthy subject is a subject not suffering from a IGF1-R kinase related disorder or disease.

“Inhibitors,” “activators,” and “modulators” of expression or of activity are used to refer to inhibitory, activating, or modulating molecules, respectively, identified using in-vitro and in-vivo assays for expression or activity. Inhibitors of the present invention are compositions that, inhibit expression of IGF1-R kinase or bind to, partially or totally block stimulation, decrease, prevent, delay activation, inactivate, desensitize, or down regulate the activity of IGF1-R kinase. Samples or assays comprising IGF1-R kinase can be treated with a composition of the present invention and compared to control samples without a composition of the present invention. Control samples (untreated with compositions of the present invention) can be assigned a relative activity value of 100%. In certain embodiments, inhibition of IGF1-R kinase is achieved when the activity value relative to the control is about 80% or less, optionally 50% or 25, 10%, 5% or 1%.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects such as nausea, dizziness, gastric upset and the like which would be to a degree that would prohibit administration of the compound.

A “therapeutically effective amount” or “pharmaceutically effective amount” means the amount that, when administered to a subject, produces effects for which it is administered. For example, a “therapeutically effective amount,” when administered to a subject to inhibit IGF1-R kinase activity, is sufficient to inhibit IGF1-R kinase activity. A “therapeutically effective amount,” when administered to a subject for treating a disease, is sufficient to effect treatment for that disease.

Except when noted, the terms “subject” or “patient” are used interchangeably and refer to mammals such as human patients and non-human primates, as well as experimental animals such as rabbits, rats, and mice, and other animals. Accordingly, the term “subject” or “patient” as used herein means any mammalian patient or subject to which the compounds of the invention can be administered. In an exemplary embodiment of the present invention, to identify subject patients for treatment according to the methods of the invention, accepted screening methods are employed to determine risk factors associated with a targeted or suspected disease or condition or to determine the status of an existing disease or condition in a subject. These screening methods include, for example, conventional work-ups to determine risk factors that are associated with the targeted or suspected disease or condition. These and other routine methods allow the clinician to select patients in need of therapy using the methods and formulations of the present invention.

The present invention provides compounds of formula I as pharmaceuticals. In a preferred embodiment, compounds of formula I are formulated as pharmaceuticals to treat diseases associated with IGF1-R kinase activity, e.g., by inhibiting growth of cancerous cell lines, including but limited to for example, the growth of human breast carcinoma in a subject.

In general, the compounds of formula I can be administered as pharmaceutical compositions by any method known in the art for administering therapeutic drugs including oral, buccal, topical, systemic (e.g., transdermal, intranasal, or by suppository), or parenteral (e.g., intramuscular, subcutaneous, or intravenous injection). Compositions can take the form of tablets, pills, capsules, semisolids, powders, sustained release formulations, solutions, suspensions, emulsions, syrups, elixirs, aerosols, or any other appropriate compositions; and comprise at least one compound of this invention in combination with at least one pharmaceutically acceptable excipient. Suitable excipients are well known to persons of ordinary skill in the art, and they, and the methods of formulating the compositions, can be found in such standard references as Alfonso AR: Remington's Pharmaceutical Sciences, 17th ed., Mack Publishing Company, Easton Pa., 1985. Suitable liquid carriers, especially for injectable solutions, include water, aqueous saline solution, aqueous dextrose solution, and glycols. In some embodiments of the present invention, the 2-arylthiazolyl compounds of Formula I suitable for use in the practice of this invention will be administered either singly or in combination with at least one other compound of this invention. The compounds of formula I suitable for use in the practice of the present invention can also be administered with at least one other conventional therapeutic agent for the disease being treated. Compounds of the invention may preferably be orally administered, for example, with an inert diluent or with an assimilable edible carrier, or they may be enclosed in hard or soft shell gelatin capsules, or they may be compressed into tablets or they may be incorporated directly with the food of the diet. For oral therapeutic administration, these active compounds may be incorporated with excipients and used in the form of ingestible tablets, buccal tablets, troches, capsules, elixirs, suspensions, syrups, wafers and the like. Such compositions and preparations should contain at least 0.1% of active compound. The percentage of the compositions and preparations may, of course, be varied and may conveniently be between about 2% to about 60% of the weight of the unit. The amount of active compound in such therapeutically useful compositions is such that a suitable dosage will be obtained. Preferred compositions or preparations according to the present invention are prepared so that an oral dosage unit form contains between 10 and 1000 mg of active compound.

Aqueous suspensions of the invention can contain a compound of formula I in admixture with excipients suitable for the manufacture of aqueous suspensions. Such excipients can include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents, and one or more sweetening agents, such as sucrose, aspartame or saccharin. Formulations can be adjusted for osmolarity.

Oil suspensions can be formulated by suspending a compound of formula I in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. Under ordinary conditions of storage and use, these preparations contain a preservative to prevent the growth or microorganisms.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. Where the compounds are sufficiently soluble they can be dissolved directly in normal saline with or without the use of suitable organic solvents, such as propylene glycol or polyethylene glycol. Dispersions of the finely divided compounds can be made-up in aqueous starch or sodium carboxymethyl cellulose solution, or in suitable oil, such as arachis oil. These formulations can be sterilized by conventional, well-known sterilization techniques. The formulations can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compound of formula I in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol. The formulations of compounds of formula I can be presented in unit-dose or multi-dose sealed containers, such as ampules and vials. Injection solutions and suspensions can be prepared from sterile powders, granules, and tablets of the kind previously described.

The compounds of this invention can be administered orally. The amount of a compound of the present invention in the composition can vary widely depending on the type of composition, size of a unit dosage, kind of excipients, and other factors well known to those of ordinary skill in the art. In general, the final composition can comprise from, for example, 0.000001 percent by weight (% w) to 10% w of the compound of formula 1, preferably 0.00001% w to 1% w, with the remainder being the excipient or excipients.

The compounds of the present invention can also be administered in the form of suppositories for rectal administration of the drug. These formulations can be prepared by mixing the drug with a suitable non-irritating excipient, which is solid at ordinary temperatures but liquid at the rectal temperatures and will therefore melt in the rectum to release the drug. Such materials are cocoa butter and polyethylene glycols.

The compounds of the present invention can also be administered by intranasal, intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995).

The compounds of formula I can be delivered transdermally, by a topical route, formulated as applicator sticks, solutions, suspensions, emulsions, gels, creams, ointments, pastes, jellies, paints, powders, and aerosols.

Encapsulating materials can also be employed with the compounds of the present invention and the term “composition” can include the active ingredient in combination with an encapsulating material as a formulation, with or without other carriers. For example, the compounds of the present invention can also be delivered as microspheres for slow release in the body. In one embodiment, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao, Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months. Cachets can also be used in the delivery of the compounds of the present invention, e.g., anti-atherosclerotic medicaments.

In another embodiment, the compounds of the present invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compound into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). In other cases, the preferred preparation can be a lyophilized powder which may contain, for example, any or all of the following: 1 mM-50 mM histidine, 0.1%-2% sucrose, 2%-7% mannitol, at a pH range of 4.5 to 5.5, that is combined with buffer prior to use.

A pharmaceutical composition of the invention can optionally contain, in addition to a compound of formula I, at least one other therapeutic agent useful in the treatment of a disease or condition associated with IGFR kinase activity. The pharmaceutical compositions are generally formulated as sterile, substantially isotonic and in full compliance with all Good Manufacturing Practice (GMP) regulations of the U.S. Food and Drug Administration. The pharmaceutical compositions of the present invention may comprise combining compounds of the invention with one or more other kinase-inhibiting compounds or chemotherapeutic agents. Chemotherapeutic agents include, but are not limited to exemestane, formestane, anastrozole, letrozole, fadrozole, taxane and derivatives such as paclitaxel or docetaxel, encapsulated taxanes, CPT-11, camptothecin derivatives, anthracycline glycosides, e.g., doxorubicin, idarubicin, epirubicin, etoposide, navelbine, vinblastine, carboplatin, cisplatin, estramustine, celecoxib, tamoxifen, raloxifen, Sugen SU-5416, Sugen SU-6668, and Herceptin. Methods of administrating a pharmaceutical composition in accordance with the invention are not specifically restricted, and can be administered in various preparations depending on the age, sex, and symptoms of the patient. For example, tablets, pills, solutions, suspensions, emulsions, granules and capsules may be orally administered. Injection preparations may be administered individually or mixed with injection transfusions such as glucose solutions and amino acid solutions intravenously. If necessary, the injection preparations are administered singly intramuscularly, intracutaneously, subcutaneously or intraperitoneally. Suppositories may be administered into the rectum. The dosage of a pharmaceutical composition according to the present invention will depend on the method of use, the age, sex, and condition of the patient.

The terms “cancer” and “cancerous” refer to or describe the physiological condition in mammals that is typically characterized by unregulated cell growth. A “tumor” comprises one or more cancerous cells. Examples of cancer treated by compounds of the present invention include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatiá cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. For the treatment of cancer, the compounds of this invention may be administered in combination with other antitumor substances or with radiation therapy. These other substances or radiation treatments may be given at the same or at different times as the compounds of this invention. These combined therapies may effect synergy and result in improved efficacy. For example, the compounds of this invention may be used in combination with mitotic inhibitors such as taxol or vinblastine, alkylating agents such as cisplatin or cyclophosamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, and antiestrogens such as tamoxifen.

The present invention provides methods of inhibiting IGFR kinase activity in a subject for the treatment of diseases and conditions associated with IGFR kinase activity using a compound of the invention. In an exemplary embodiment of the present invention, a skilled practitioner will treat a subject having a disease associated with IGF1-R kinase activity with the compounds of the present invention.

For treatment purposes, the compositions or compounds disclosed herein can be administered to the subject in a single bolus delivery, via continuous delivery (e.g., continuous transdermal, mucosal, or intravenous delivery) over an extended time period, or in a repeated administration protocol (e.g., by an hourly, daily or weekly, repeated administration protocol). The pharmaceutical formulations of the present invention can be administered, for example, one or more times daily, 3 times per week, or weekly. In an exemplary embodiment of the present invention, the pharmaceutical formulations of the present invention are orally administered once or twice daily.

In this context, a therapeutically effective dosage of the biologically active agent(s) can include repeated doses within a prolonged treatment regimen that will yield clinically significant results to alleviate one or more symptoms or detectable conditions associated with increased securin activity. Determination of effective dosages in this context is typically based on animal model studies followed up by human clinical trials and is guided by determining effective dosages and administration protocols that significantly reduce the occurrence or severity of targeted exposure symptoms or conditions in the subject. Suitable models in this regard include, for example, murine, rat, porcine, feline, non-human primate, and other accepted animal model subjects known in the art. Alternatively, effective dosages can be determined using in-vitro models (e.g., immunologic and histopathologic assays). Using such models, only ordinary calculations and adjustments are typically required to determine an appropriate concentration and dose to administer a therapeutically effective amount of the biologically active agent(s) (e.g., amounts that are intranasally effective, transdermally effective, intravenously effective, or intramuscularly effective to elicit a desired response). In alternative embodiments, an “effective amount” or “therapeutically effective dose” of the biologically active agent(s) will simply inhibit or enhance one or more selected biological activity(ies) correlated with a disease or condition, as set forth above, for either therapeutic or diagnostic purposes.

The actual dosage of biologically active agents will of course vary according to factors such as the extent of exposure and particular status of the subject (e.g., the subject's age, size, fitness, extent of symptoms, susceptibility factors, etc), time and route of administration, as well as other drugs or treatments being administered concurrently. Dosage regimens can be adjusted to provide an optimum prophylactic or therapeutic response. By “therapeutically effective dose” herein is meant a dose that produces effects for which it is administered. More specifically, a therapeutically effective dose of the compound(s) of the invention preferably alleviates symptoms, complications, or biochemical indicia of diseases associated with increased securin activity. The exact dose will depend on the purpose of the treatment, and will be ascertainable by one skilled in the art using known techniques (see, e.g., Lieberman, Pharmaceutical Dosage Forms (Vols. 1-3, 1992); Lloyd, 1999, The Art, Science, and Technology of Pharmaceutical Compounding; and Pickar, 1999, Dosage Calculations). A therapeutically effective dose is also one in which any toxic or detrimental side effects of the active agent is outweighed in clinical terms by therapeutically beneficial effects. It is to be further noted that for each particular subject, specific dosage regimens should be evaluated and adjusted over time according to the individual need and professional judgment of the person administering or supervising the administration of the compound.

In an exemplary embodiment of the present invention, unit dosage forms of the compounds are prepared for standard administration regimens. In this way, the composition can be subdivided readily into smaller doses at the physicians direction. For example, unit dosages can be made up in packeted powders, vials or ampoules and preferably in capsule or tablet form. The active compound present in these unit dosage forms of the composition can be present in an amount of, for example, from about one gram to about fifteen grams or more, for single or multiple daily administration, according to the particular need of the patient. By initiating the treatment regimen with a minimal daily dose of about one gram, the blood levels of securin and the patients symptomatic relief analysis can be used to determine whether a larger or smaller dose is indicated. Effective administration of the compounds of this invention can be given at an oral dose of from, for example about 0.1 mg/kg/day to about 1,000 mg/kg/day. Preferably, administration will be from about 10/mg/kg/day to about 600 mg/kg/day, more preferably from about 25 to about 200 mg/kg/day, and even more preferably from about 50 mg/kg/day to about 100 mg/kg/day.

In certain embodiments, the present invention is directed to prodrugs of compounds of formula I. The term “prodrug,” as used herein, means a compound that is convertible in vivo by metabolic means (e.g. by hydrolysis) to a compound of formula I. Various forms of prodrugs are known in the art such as those discussed in, for example, Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991), Bundgaard, et al., Journal of Drug Delivery Reviews, 8:1-38 (1992), Bundgaard, J. of Pharmaceutical Sciences, 77:285 et seq. (1988); and Higuchi and Stella (eds.) Prodrugs as Novel Drug Delivery Systems, American Chemical Society (1975).

After a pharmaceutical comprising a compound of formula I has been formulated in a suitable carrier, it can be placed in an appropriate container and labeled for treatment of a securin related disorder, e.g., breast cancer. Additionally, another pharmaceutical comprising at least one other therapeutic agent useful in the treatment of the securin related disorder can be placed in the container as well and labeled for treatment of the indicated disease. Alternatively, a single pharmaceutical comprising a compound of formula I and at least one other therapeutic agent useful in the treatment of an IGFR kinase related disorder can be placed in an appropriate container and labeled for treatment. For administration of pharmaceuticals comprising a compound of formula I and of pharmaceuticals comprising, in a single pharmaceutical, a compound of formula I and at least one other therapeutic agent useful in the treatment of a securin related disorder, such labeling would include, for example, instructions concerning the amount, frequency and method of administration. Similarly, for administration of multiple pharmaceuticals provided in the container, such labeling would include, for example, instructions concerning the amount, frequency and method of administration of each pharmaceutical.

Based on the results of standard pharmacological test procedures described herein, the compounds of the invention are useful as agents for treating, inhibiting or controlling the growth of cancerous tumor cells and associated diseases in a mammal in need thereof. The compounds of the invention are useful as agents for treating, inhibiting or controlling the growth of cancerous tumor cells and associated diseases in a mammal. In the case of cancer treatment, it is believed that many neoplasias such as leukemia, lung cancer, colon cancer, thyroid cancer, ovarian cancer, renal cancer, prostate cancer and breast cancers may be treated by effectively administering effective amounts of a compound of formula I. Suitable examples of cancers for treatment using methods provided herein include carcinoma, sarcoma, lymphoma, or leukemia. The term “carcinoma” refers to a benign or malignant epithelial tumor and includes, but is not limited to, breast carcinoma, prostate carcinoma, non-small lung carcinoma, colon carcinoma, melanoma carcinoma, ovarian carcinoma, or renal carcinoma. A preferred subject or mammalian host benefiting from treatment using one or more compounds of the invention is a human.

Selected compounds of formula I were tested as inhibitors of IGF1-R kinase activity. The biological activity range of Examples 1-30, as evidenced by IC₅₀ values, is: 0.002 μM<x<20 μM.

Examples of compounds of formula I were evaluated in several standard pharmacological test procedures that showed that the compounds of this invention inhibit IGFR kinase activity. Based on the activity shown in the standard pharmacological test procedures, the compounds of this invention are therefore useful as anti-cancer agents. Associated cancers are selected from the group consisting of breast, colon, lung, prostate, melanoma, epidermal, leukemia, kidney, bladder, mouth, larynx, esophagus, stomach, ovary, pancreas, liver, skin and brain. In particular, the compounds of this invention possess an effect similar to HKI-272. The test procedures used and results obtained are shown below. Having described the invention, the invention is further illustrated by the following non-limiting examples.

### EXAMPLES

### Anilino-group 1: 3-Chloro-4-(1-ethyl-4,5-dimethyl-1H-imidazol-2-ylsulfanyl)-phenylamine

### Step 1. 2-(4-Methoxy-benzylsulfanyl)-4,5-dimethyl-1H-imidazole

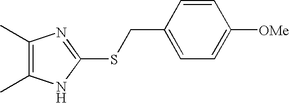

To a stirred solution of 4,5-dimethyl-1H-imidazole-2-thiol (2.00 g, 15.6 mmol) in NMP (50 mL) was added DIEA (2.72 mL, 16.4 mmol) and 5-methoxybenzyl chloride (2.72 mL, 16.4 mmol). The reaction mixture was stirred at rt overnight, diluted with H₂O, and the precipitate was collected via vacuum filtration to give the desired product (3.14 g, 12.6 mmol, 81%) as a white solid; ¹H NMR (DMSO-d₆) δ 1.97 (2, 6H), 3.67 (s, 3H), 4.06 (s, 2H), 6.78 (d, J=8.7 Hz, 2H), 7.14 (d, J=8.7 Hz, 2H), 11.67 (s, 1H); mass spectrum [(+) ESI], m/z 249 (M+H).

### Step 2. 1-Ethyl-2-(4-methoxy-benzylsulfanyl)-4,5-dimethyl-1H-imidazole

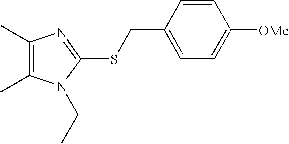

To a stirred solution of 2-(4-methoxy-benzylsulfanyl)-4,5-dimethyl-1H-imidazole (3.14 g, 12.6 mmol) in DMF (30 mL) at 0° C. was added NaH (0.56 g, 13.9 mmol). After stirring for 15 minutes at 0° C., iodoethane (1.11 mL, 13.9 mmol) was added dropwise. The reaction mixture was stirred an additional 15 minutes at 0° C., then warmed to rt and stirred overnight. The reaction was quenched with MeOH (20 mL), diluted with H₂O (10 mL) and extracted with EtOAc (3×50 mL). The organic layer was washed with brine (50 mL), then dried over MgSO₄, filtered and concentrated. The residue was purified by column chromatography (0 to 5% MeOH:CHCl₃ gradient) to afford the product (2.9 g, 12.6 mmol, 83%) as an oil; ¹H NMR (DMSO-d₆) δ 0.96 (t, J=14.5 Hz, 3H), 1.97-2.03 (m, 2H), 2.69 (s, 3H), 2.84 (s, 3H), 3.67 (s, 3H), 4.04 (s, 2H), 6.78 (d, J=8.7 Hz, 2H), 7.10 (d, J=8.7 Hz, 2H), 11.64 (s, 1H); mass spectrum [(+) ESI], m/z 277 (M+H).

### Step 3. 1-Ethyl-4,5-dimethyl-1H-imidazole-2-thiol

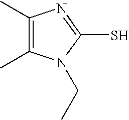

To a stirred solution of 1-ethyl-2-(4-methoxy-benzylsulfanyl)-4,5-dimethyl-1H-imidazole (3.48 g, 12.6 mmol) in toluene (10 mL) was added TFA (10 mL). The reaction mixture was heated at 100° C. for 60 hours. The solution was concentrated and purified by column chromatography (0 to 5% MeOH: CHCl₃ gradient) to afford the product (1.51 g, 9.66 mmol, 77%) as an oil; ¹H NMR (DMSO-d₆) δ 1.09 (t, J=14.3 Hz, 3H), 1.96 (s, 3H), 2.06 (s, 3H), 3.88 (q, J=19.5 Hz, 2H), 7.90 (s, 1H); mass spectrum [(+) ESI], m/z 157 (M+H).

### Step 4. 2-(2-chloro-4-nitro-phenylsulfanyl)-1-ethyl-4,5-dimethyl-1H-imidazole

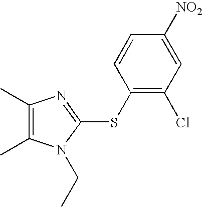

To a stirred solution of 1-ethyl-4,5-dimethyl-1H-imidazole-2-thiol (1.51 g, 9.66 mmol) in DMF (20 mL) at 0° C. was added NaH (0.425 g, 10.6 mmol). After 15 min at 0° C., 3-chloro-4-fluoronitrobenzene (0.1.70 g, 9.66 mmol) was added slowly, the reaction mixture was stirred at 0° C. 30 min, warmed to room temperature and stirred overnight. The reaction was diluted with H₂O (20 mL) and extracted with EtOAc (3×50 mL). The organic layer was washed with brine (50 mL), dried over MgSO₄, filtered, and then concentrated. The residue was purified by column chromatography (0 to 10% MeOH:CHCl₃ gradient) to afford the product (2.2 g, 7.06 mmol, 73%) as an oil; ¹H NMR (DMSO-d₆) δ 1.06 (t, J=14.5 Hz, 3H), 2.10 (s, 3H), 2.18 (s, 3H), 3.88 (q, J=21.7 Hz, 2H), 6.61 (d, J=8.9 Hz, 1H), 8.04 (dd, J=2.4, 8.9 Hz, 1H), 8.29 (s, 1H); mass spectrum [(+) ESI], m/z 312 (M+H).

### Step 5. 3-Chloro-4-(1-ethyl-4,5-dimethyl-1H-imidazol-2-ylsulfanyl)-phenylamine

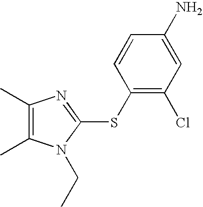

To a stirred solution of 2-(2-chloro-4-nitro-phenylsulfanyl)-1-ethyl-4,5-dimethyl-1H-imidazole (2.20 g, 7.06 mmol) in MeOH:H₂O (5:1, 54 mL) was added iron (1.24 g, 22.3 mmol) followed by NH₄Cl (1.24 g, 34.2 mmol). The reaction mixture was heated to reflux for 2 hours, cooled to room temperature, then diluted with H₂O (20 mL) and EtOAc (60 mL), filtered through celite and washed with EtOAc. The layers were separated, and the organic layer was washed with brine (60 mL), dried over MgSO₄, filtered and then concentrated. The residue was purified by column chromatography (0 to 5% MeOH:CHCl₃) to afford the product (1.4 g, 4.97 mmol, 71%) as a solid; ¹H NMR (DMSO-d₆) δ 0.98 (t, J=14.5 Hz, 3H), 1.99 (s, 3H), 2.08 (s, 3H), 3.89 (q, J=21.7 Hz, 2H), 5.47 (s, 2H), 6.68-6.74 (m, 3H); mass spectrum [(+) ESI], m/z 282 (M+H).

### Anilino-group 2: 3-Chloro-4-(4,5-dimethyl-1-propyl-1H-imidazol-2-ylsulfanyl)-phenylamine

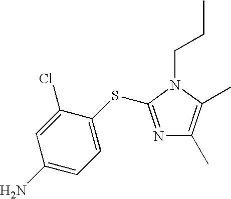

This anilino-group was prepared as a solid (0.364 g, 16% for 5 steps) from propyl iodide in step one, using a procedure similar to steps 1-5 of Anilino-group 1, ¹H NMR (DMSO-d₆) 6) 0.71-0.78 (m, 3H), 1.37-1.46 (m, 2H), 1.99 (s, 3H), 2.03 (s, 3H), 3.80 (q, J=22.0 Hz, 2H), 5.47 (s, 2H), 6.38 (dd, J=2.3, 10.9 Hz, 1H), 6.49 (dd, J=2.3, 10.9 Hz, 1H), 6.72 (dd, J=2.3, 10.9 Hz, 1H); mass spectrum [(+) ESI], m/z 296 (M+H).

### Anilino-group 3: 3-Chloro-4-(1-isopropyl-4,5-dimethyl-1H-imidazol-2-ylsulfanyl)-phenylamine

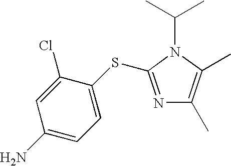

This anilino-group was prepared as a solid (0.297 g, 22% for 5 steps) from isopropyl iodide in step one, using a procedure similar to steps 1-5 of Anilino-group 1, ¹H NMR (DMSO-d₆) 6) 1.26 (d, J=6.9 Hz, 6H), 1.97 (s, 3H), 2.17 (s, 3H), 4.72-4.79 (m, 1H), 5.45 (s, 2H), 6.50 (dd, J=2.4, 8.6 Hz, 1H), 6.60-6.72 (m, 2H); mass spectrum [(+) ESI], m/z 296 (M+H).

### Anilino-groups 4-8: 4-(1-benzyl-4,5-dimethyl-1H-imidazol-2-ylthio)-3-chloroaniline, 3-chloro-4-(1,5-dimethyl-1H-benzo[d]imidazol-2-ylthio)aniline, 3-chloro-4-(1,4,5-trimethyl-1H-imidazol-2-ylsulfanyl)-phenylamine, 3-bromo-4-(1,4,5-trimethyl-1H-imidazol-2-ylthio)aniline, 4-(1,4,5-trimethyl-1H-imidazol-2-ylthio)aniline were prepared using a procedure similar to steps 1-5 of Anilino-group 1.

### Example 1

### 4-({3-Chloro-4-[(1,4,5-trimethyl-1H-imidazol-2-yl)thio]phenyl}amino-7-{[3-(dimethylamino)propyl]amino}-6-methoxyquinoline-3-carbonitrile

### Step 1. 7-Fluoro-4-hydroxy-6-methoxy-quinoline-3-carbonitrile

To a stirred solution of 3-fluoro-p-anisidine (5.00 g, 35.4 mmol) in toluene (10 mL) was added ethyl (ethoxymethylene)-cyanoacetate (5.99 g, 35.4 mmol), and the solution was heated to 100° C. for 1 hour, followed by 125° C. for 0.5 hour. The mixture was concentrated, and the residue was recrystallized from EtOAc:hexane. This intermediate solid was taken up in Dowtherm A (200 mL) and transferred to a 3-neck flask equipped with an air condenser, bubbler and thermometer. The solution was heated at 250° C. overnight, cooled to room temperature, diluted with hexanes (600 mL) and the brown precipitate was collected via vacuum filtration and then washed with hexanes followed by CH₂Cl₂ to afford the product (4.86 g, 63%) as a solid; ¹H NMR (DMSO-d₆) δ 3.91 (s, 3H), 7.42 (d, J=11.5 Hz, 1H), 7.64 (d, J=9.2 Hz, 1H), 8.65 (s, 1H), 12.74 (s, 1H); mass spectrum [(+) ESI], m/z 219 (M+H).

### Step 2. 4-Chloro-7-fluoro-6-methoxy-quinoline-3-carbonitrile

A stirred solution of 7-fluoro-4-hydroxy-6-methoxy-quinoline-3-carbonitrile (4.86 g, 22.3 mmol) in POCl₃ (20 mL) with a few drops DMF (˜30) was heated to 110° C. for 4 hours. After cooling to room temperature, the solution was poured over ice/H₂O and neutralized with saturated NaHCO₃. The precipitate was collected via vacuum filtration, washed with H₂O and then absorbed onto silica. It was purified by column chromatography (5 to 40% EtOAc: petroleum ether gradient) to afford the product (2.42 g, 46%) as a solid; ¹H NMR (DMSO-d₆) δ 4.06 (s, 3H), 7.65 (d, J=8.9 Hz, 1H), 8.02 (d, J=11.8 Hz, 1H), 9.05 (s, 1H); mass spectrum [(+) ESI], m/z 237 (M+H).

### Step 3. 4-({3-Chloro-4-[(1,4,5-trimethyl-1H-imidazol-2-yl)thio]phenyl}-amino-7-fluoro-6-methoxy-quinoline-3-carbonitrile

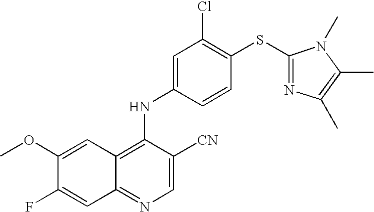

To a stirred solution of 3-chloro-4-(1,4,5-trimethyl-1H-imidazol-2-ylsulfanyl)-phenylamine (0.057 g, 0.211 mmol) in ethoxyethanol (2 mL) was added 4-chloro-7-fluoro-6-methoxy-quinoline-3-carbonitrile (0.050 g, 0.211 mmol) and pyridine HCl (0.027 g, 0.232 mmol). The solution was heated at 130° C. for 2 hours, cooled to room temperature and then quenched with saturated NaHCO₃ (5 mL). This mixture was stirred for 15 minutes. The resulting precipitate was collected via vacuum filtration and then washed with H₂O and Et₂O to afford the product (0.049 g, 50%) as a solid; ¹H NMR (DMSO-d₆) δ 2.06 (s, 3H), 2.12 (s, 3H), 3.39 (s, 3H), 3.94 (s, 3H), 6.51 (d, J=8.5 Hz, 1H), 7.09 (d, J=9.0 Hz, 1H), 7.36 (s, 1H), 7.68 (d, J=12.2 Hz, 1H), 7.90 (d, J=9.3 Hz, 1H), 8.45 (s, 1H), 9.65-9.73 (bs, 1H); mass spectrum [(+) ESI], m/z 468 (M+H).

### Step 4. 4-({3-Chloro-4-[(1,4,5-trimethyl-1H-imidazol-2-yl)thio]phenyl}-amino-7-{[3-(dimethylamino)propyl]amino}-6-methoxyquinoline-3-carbonitrile

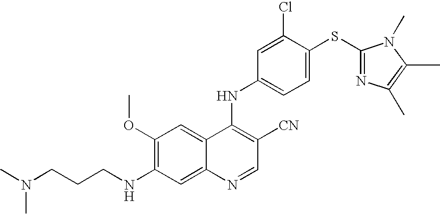

To a stirred solution of 4-({3-chloro-4-[(1,4,5-trimethyl-1H-imidazol-2-yl)thio]phenyl}amino-7-fluoro-6-methoxy-quinoline-3-carbonitrile (0.049 g, 0.105 mmol) in NMP (2 mL) was added 3-(dimethylamino)propylamine (0.132 mL, 1.05 mmol). The solution was heated overnight at 115° C., cooled to room temperature, then diluted with H₂O (5 mL) and extracted with EtOAc (50 mL). The organic layer was washed with brine (5 mL), dried over MgSO₄, filtered and then concentrated. The residue was triturated with Et₂O and then purified by preparative thin layer chromatography (85:10:5 CH₂Cl₂:MeOH:NH3 eluent) to afford the title compound as a solid (0.010 g, 17%); ¹H NMR (DMSO-d₆) δ 1.70 (t, J=6.4 Hz, 2H), 2.05 (s, 3H), 2.09-2.13 (m, 9H), 2.28 (t, J=6.3 Hz, 2H), 3.20-3.26 (m, 2H), 3.40 (s, 3H), 3.87 (s, 3H), 6.43-6.47 (m, 1H), 6.53 (d, J=8.6 Hz, 1H), 6.75 (s, 1H), 6.96 (dd, J=2.4, 8.7 Hz, 1H), 7.19 (d, J=2.3 Hz, 1H), 7.38 (s, 1H), 8.36 (s, 1H), 9.21 (s, 1H); mass spectrum [(+) ESI], m/z 550/552 (M+H).

### Examples 2-6

The compounds were prepared using the indicated aniline and the procedure outlined in steps 3-4 of Example 1, as summarized in Table 1.

TABLE 1
Compounds of formula

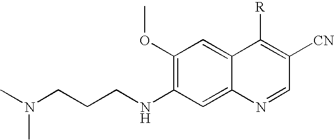

| | | Mass spectrum |
|---|---|---|
| Exam- | | [(+) ESI]: m/z |
| ple | R | (M + H) |
| 2 | 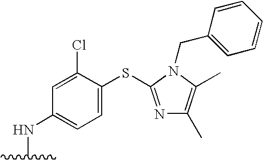 | 626/628 |
| 3 | 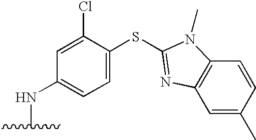 | 586/588 |
| 4 | 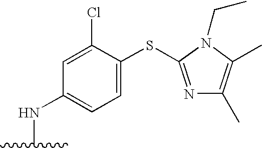 | 564/566 |
| 5 | 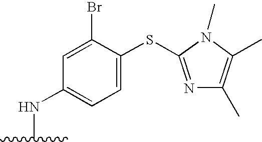 | 594/596 |
| 6 | 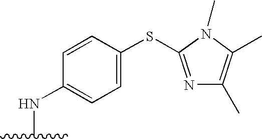 | 516 |

### Example 7

### 4-({3-Chloro-4-[(1-ethyl-4,5-dimethyl-1H-imidazol-2-yl)thio]phenyl}amino)-6-methoxy-7-[(3-phenylpropyl)amino]quinoline-3-carbonitrile

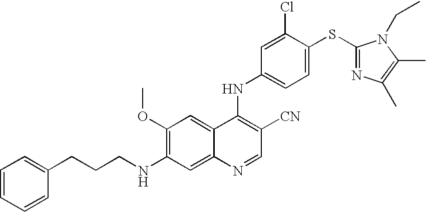

To a stirred solution of 4-({3-chloro-4-[(1-ethyl-4,5-dimethyl-1H-imidazol-2-yl)thio]phenyl}amino-7-fluoro-6-methoxy-quinoline-3-carbonitrile (0.040 g, 0.083 mmol) in NMP (2 mL) was added 3-phenyl-1-propylamine (0.118 mL, 0.830 mmol). The solution was heated at 115° C. for 6 hours and then at 100° C. overnight. After cooling to room temperature, the reaction mixture was then diluted with H₂O (˜2 mL). The resulting solid was filtered off and washed with excess H₂O to afford the title compound (0.0118 g, 24%); ¹H NMR (DMSO-d₆) δ 1.06 (t, J=7.3 Hz, 3H), 1.86-1.96 (m, 2H), 2.08 (s, 3H), 2.16 (s, 3H), 2.67 (t, J=7.4 Hz, 2H), 3.18-3.27 (m, 2H), 3.89 (s, 3H), 3.89-3.96 (m, 2H), 6.18-6.26 (m, 1H), 6.60 (d, J=8.6 Hz, 1H), 6.74 (s, 1H), 6.99 (dd, J=2.4, 11.0 Hz, 1H), 7.13-7.30 (m, 6H), 7.42 (s, 1H), 8.38 (s, 1H), 9.24 (s, 1H); mass spectrum [(+) ESI]: m/z 597/599 (M+H).

### Examples 8-9

These compounds were prepared using the indicated amine and the procedure outlined in Example 7, as summarized in Table 2.

TABLE 2
Compounds of formula:

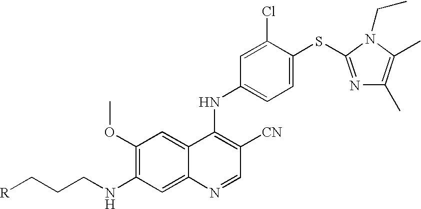

| | | Mass spectrum |
|---|---|---|
| | | [(+) ESI]: m/z |
| Example | R | (M + H) |
| 8 | 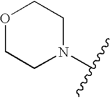 | 606/608 |
| 9 | 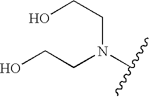 | 624/626 |

### Example 10

### N-(3-{4-[3-chloro-4-(1-ethyl-4,5-dimethyl-1H-imidazol-2-ylsulfanyl)-phenylamino]-3-cyano-6-methoxy-quinolin-7-yloxy}-propyl)-benzenesulfonamide

### Step 1. Methyl 4-(3-chloropropoxy)-3-methoxybenzoate

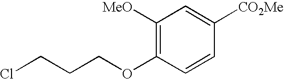

To a stirred solution of methyl vanillate (2.00 g, 11.0 mmol) in acetone (50 mL) was added Cs₂CO₃ (3.94 g, 12.1 mmol) followed by 3-chloro-1-propyl bromide (2.16 mL, 22.0 mmol). The solution was stirred at room temperature for 3 days. The solution was then diluted with H₂O (50 mL) and extracted with Et₂O (2×200 mL). The organic layer was washed with brine, dried over MgSO₄, filtered and then concentrated to afford the product (0.290 g, 85%) as a solid; ¹H NMR (DMSO-d₆) δ 2.10-2.20 (m, 2H), 3.74 (t, J=6.5 Hz, 2H), 3.77 (s, 6H), 4.12 (t, J=6.0 Hz, 2H), 7.06 (d, J=8.5 Hz, 1H), 7.41 (d, J=2.0 Hz, 1H), 7.53 (dd, J=2.1, 8.5 Hz, 1H); mass spectrum [(+) ESI], m/z 259 (M+H).

### Step 2. Methyl 4-(3-chloropropoxy)-5-methoxy-2-nitrobenzoate

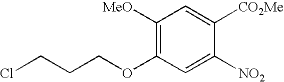

To a cooled flask of HNO₃ (12 mL) at 0° C. was slowly added methyl 4-(3-chloropropoxy)-3-methoxybenzoate (2.75 g, 10.6 mmol) portion wise over 15 minutes. The solution was stirred at this temperature for 0.5 hour and then at room temperature for 1 hour. The solution was cooled back down to 0° C. and diluted H₂O (˜10 mL). The mixture was stirred for 0.5 hour, and a yellowish orange oily solid came out. The mixture was extracted with Et₂O, and the resulting organic layer washed with brine, dried over MgSO₄, filtered and then concentrated to afford the product (3.03 g, 94%); ¹H NMR (DMSO-d₆) δ 2.12-2.21 (m, 2H), 3.73 (t, J=6.5 Hz, 2H), 3.78 (s, 3H), 3.88 (s, 3H), 4.20 (t, J=6.1 Hz, 2H), 7.28 (s, 1H), 7.63 (s, 1H); mass spectrum [(+) ESI], m/z 326 (M+H).

### Step 3. Methyl 2-amino-4-(3-chloropropoxy)-5-methoxybenzoate

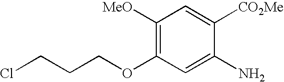

To a stirred solution of methyl 4-(3-chloropropoxy)-5-methoxy-2-nitrobenzoate (3.03 g, 9.98 mmol) in MeOH:H₂O (5:1, 48 mL) was added iron (2.23 g, 39.9 mmol) followed by NH₄Cl (2.13 g, 39.9 mmol). The solution was heated to reflux overnight. The solution was cooled to room temperature and then diluted with EtOAc (100 mL) and H₂O (30 mL). The mixture was filtered through celite, and the filtrate layers were separated. The organic layer was washed with brine, dried over MgSO₄, filtered and then concentrated. The residue was purified with Biotage Flash 40 (10% to 30% EtOAc:petroleum ether gradient) to afford the product (2.07 g, 76%); ¹H NMR (DMSO-d₆) δ 2.11-2.19 (m, 2H), 3.60 (s, 3H), 3.70 (s, 3H), 3.73 (t, J=6.5 Hz, 2H), 4.00 (t, J=6.1 Hz, 2H), 6.35 (s, 1H), 6.37 (s, 2H), 7.09 (s, 1H); mass spectrum [(+) ESI], m/z 274 (M+H).

### Step 4. (E)-Methyl 4-(3-chloropropoxy)-2-[(dimethylamino)-methyleneamino]-5-methoxybenzoate

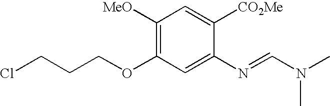

To a flask with methyl 2-amino-4-(3-chloropropoxy)-5-methoxybenzoate (2.07 g, 7.56 mmol) was added DMF-DMA (12 mL). The solution was heated to reflux for 4 hours. The solvent was remove via high vacuum, and the resulting residue was passed through florasil, eluting with CH₂Cl₂, and then concentrated to afford the product (2.47 g, 99%); ¹H NMR (DMSO-d₆) δ 2.08-2.17 (m, 2H), 2.83-2.95 (bs, 6H), 3.64 (s, 3H), 3.68 (s, 3H), 3.73 (t, J=6.5 Hz, 2H), 4.07 (t, J=6.0 Hz, 2H), 6.44 (s, 1H), 7.11 (s, 1H), 7.43 (s, 1H); mass spectrum [(+) ESI], m/z 329 (M+H).

### Step 5. 7-(3-Chloropropoxy)-4-hydroxy-6-methoxyquinoline-3-carbonitrile

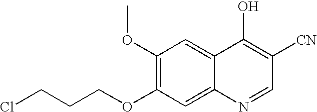

To a stirred solution of n-BuLi in hexanes (6.76 mL, 2.5 M solution) at −78° C. was added a solution of acetonitrile (0.88 mL, 16.9 mmol) in THF (20 mL) dropwise. The reaction mixture was stirred ˜15 minutes at this temperature. Then a solution of (E)-methyl 4-(3-chloropropoxy)-2-[(dimethylamino)methyleneamino]-5-methoxybenzoate (2.52 g, 7.66 mmol) in THF (20 mL) was added dropwise over 20 minutes. The mixture was kept at −78° C. for 2 hours and then removed from a dry ice bath for 0.5 hour. After replacing the dry ice bath, HOAc (2.19 mL, 38.3 mmol) was added dropwise, and the mixture was warmed to room temperature overnight. The solvent was removed via high vacuum, and the residue was diluted with H₂O (˜20 mL). The resulting yellow precipitate was filtered off and washed with excess H₂O and Et₂O to afford the product (1.90 g g, 85%); ¹H NMR (DMSO-d₆) δ 2.15-2.24 (m, 2H), 3.77 (t, J=6.5 Hz, 2H), 3.81 (s, 3H), 4.13 (t, J=6.0 Hz, 2H), 7.01 (s, 1H), 7.41 (s, 1H), 8.40 (s, 1H), 11.74-12.47 (bs, 1H); mass spectrum [(+) ESI], m/z 293 (M+H).

### Step 6. 4-Chloro-7-(3-chloropropoxy)-6-methoxyquinoline-3-carbonitrile

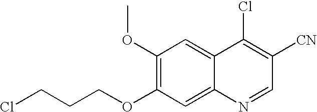

A stirred solution of 7-(3-chloropropoxy)-4-hydroxy-6-methoxyquinoline-3-carbonitrile (1.90 g, 6.49 mmol) in POCl₃ (10 mL) with a few drops DMF (˜20) was heated to 110° C. for 1 hour. After cooling to room temperature, the solution was then concentrated and azetroped with toluene. The residue was poured over ice/H₂O and neutralized with saturated NaHCO₃. The precipitate was collected via vacuum filtration, washed with excess H₂O and Et₂O. The product was purified with Biotage™ Flash 40 (20 to 40% EtOAc:petroleum ether gradient) to afford the product (1.39 g, 69%); ¹H NMR (DMSO-d₆) δ 2.21-2.29 (m, 2H), 3.78 (t, J=6.4 Hz, 2H), 3.98 (s, 3H), 4.32 (t, J=6.0 Hz, 2H), 7.42 (s, 1H), 7.54 (s, 1H), 8.95 (s, 1H); mass spectrum [(+) ESI], m/z 311/313 (M+H)⁺.

### Step 7. 4-[3-Chloro-4-(1-ethyl-4,5-dimethyl-1H-imidazol-2-ylsulfanyl)-phenylamino]-7-(3-chloro-propoxy)-6-methoxy-quinoline-3-carbonitrile

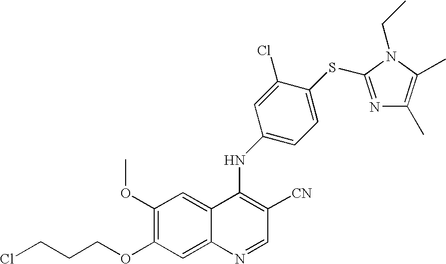

To a stirred solution of 4-chloro-7-(3-chloro-propoxy)-6-methoxy-quinoline-3-carbonitrile (0.298 g, 0.958 mmol) in ethoxyethanol (6 mL) was added 3-chloro-4-(1-ethyl-4,5-dimethyl-1H-imidazol-2-ylsulfanyl)-phenylamine (0.270 g, 0.958 mmol) and pyridine HCl (0.122 g, 1.05 mmol). The mixture was heated to 120° C., maintaining the temperature overnight. The solution was cooled to room temperature, then quenched with saturated NaHCO₃ solution (10 mL), stirred at room temperature for 30 minutes and the precipitate was collected via vacuum filtration. The solid was absorbed on silica and purified by flash chromatography (0 to 3% MeOH:CHCl₃ gradient) to afford the product (0.240 g, 0.431 mmol, 45%) as a solid; mass spectrum [(+) ESI], m/z 557 (M+H).

### Step 8. N-(3-{4-[3-chloro-4-(1-ethyl-4,5-dimethyl-1H-imidazol-2-ylsulfanyl)-phenylamino]-3-cyano-6-methoxy-quinolin-7-yloxy}-propyl)-benzenesulfonamide

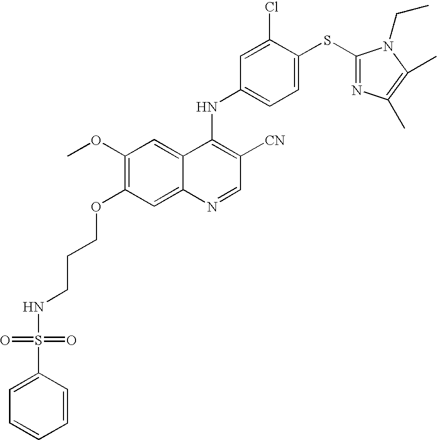

To a stirred solution of benzenesulfonamide (0.0465 g, 0.0539 mmol) in DMF (1 mL) was added NaH (0.012 g, 0.0296 mmol). The solution was stirred at rt for 30 min, then 4-[3-chloro-4-(1-ethyl-4,5-dimethyl-1H-imidazol-2-ylsulfanyl)-phenylamino]-7-(3-chloro-propoxy)-6-methoxy-quinoline-3-carbonitrile (0.030 g, 0.0539 mmol) was added, and the mixture was heated to 100° C., maintaining the temperature overnight. The solution was cooled to room temperature, diluted with brine (1 mL), then stirred at room temperature for 30 minutes. The precipitate was collected via vacuum filtration and washed with H₂O and Et₂O to afford the product (0.0246 g, 4.00 mmol, 44%) as a solid; ¹H NMR (DMSO-d₆) δ 1.04 (t, J=14.4 Hz, 3H), 1.86-1.91 (m, 2H), 2.06 (s, 3H), 2.16 (s, 3H), 2.91-2.98 (m, 2H), 3.88-3.95 (m, 5H), 4.08-4.11 (m, 2H), 6.56 (d, J=8.7 Hz, 1H), 7.07-7.09 (m, 1H), 7.22 (s, 1H), 7.34 (s, 1H), 7.52-7.58 (m, 3H), 7.75-7.81 (m, 4H), 8.44 (s, 1H), 9.97 (s, 1H); mass spectrum [(+) ESI], m/z 615 (M+H).

### Examples 11-16

These compounds were prepared using the indicated amine and the procedure outlined in step 8 of Example 10, as summarized in Table 3.

TABLE 3
Compounds of formula:

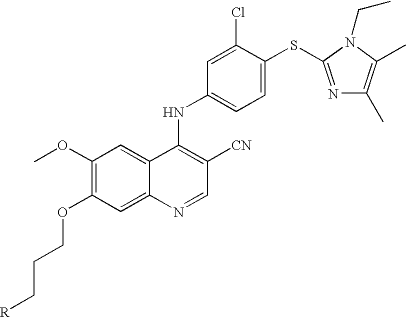

| | | Mass spectrum |
|---|---|---|
| | | [(+) ESI]: m/z |
| Example | R | (M + H) |
| 11 | 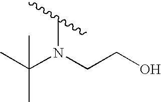 | 637 |
| 12 | 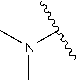 | 565 |
| 13 | 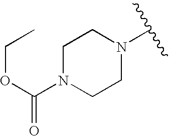 | 678 |
| 14 | 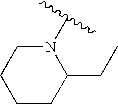 | 633 |
| 15 | 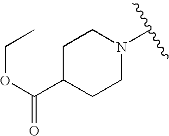 | 677 |
| 16 | 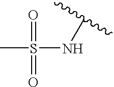 | 615 |

### Example 17

### 4-({3-Chloro-4-[(1-ethyl-4,5-dimethyl-1H-imidazol-2-yl)sulfanyl]phenyl}amino)-7-[3-(4-ethyl-1-piperazinyl)propoxy]-6-methoxy-3-quinolinecarbonitrile

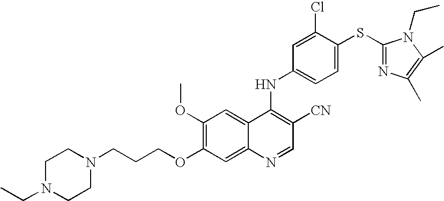

To a stirred solution of 4-[3-chloro-4-(1-ethyl-4,5-dimethyl-1H-imidazol-2-ylsulfanyl)-phenylamino]-7-(3-chloro-propoxy)-6-methoxy-quinoline-3-carbonitrile (0.300 g, 0.539 mmol) in DMF (2 mL) was added 1-ethyl-piperazine (0.137 mL, 1.08 mmol) and NaI (0.008 g, 0.0539 mmol). The solution was heated to 100° C., maintaining the temperature overnight. The solution was cooled to room temperature and then diluted with brine (2 mL) and H₂O (2 mL). The precipitate was collected via vacuum filtration and washed with H₂O and Et₂O to afford the product (0.290 g, 85%) as a solid, mp. 229-230° C.; ¹H NMR (DMSO-d₆) δ 0.93 (t, J=7.2 Hz, 3H), 1.04 (t, J=7.1 Hz, 3H), 1.85-1.95 (m, 2H), 2.07 (s, 3H), 2.15 (s, 3H), 2.22-2.43 (m, 12H), 3.86 (s, 3H), 3.90 (dd, J=7.1, 14.4 Hz, 2H), 4.14 (t, J=6.4 Hz, 2H), 6.58 (d, J=8.6 Hz, 1H), 7.06 (dd, J=2.3, 8.7 Hz, 1H), 7.29 (s, 1H), 7.30 (d, J=2.3 Hz, 1H), 7.61 (s, 1H), 8.45 (s, 1H), 9.46 (s, 1H); mass spectrum [(+) ESI], m/z 634/636 (M+H).

### Examples 18-27

These compounds were prepared using the indicated amine and the procedure outlined in Example 17, as summarized in Table 4.

TABLE 4
Compounds of formula:

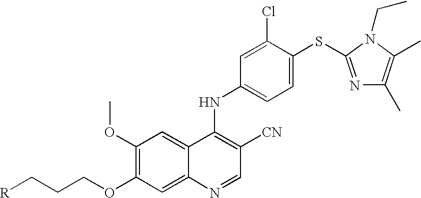

| | | Mass spectrum |
|---|---|---|
| | | [(+) ESI]: m/z |
| Example | R | (M + H) |
| 18 | 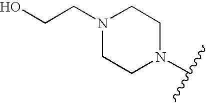 | 650/652 |
| 19 | 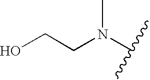 | 595/597 |
| 20 | 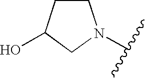 | 607/609 |
| 21 | 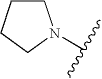 | 591/593 |
| 22 | 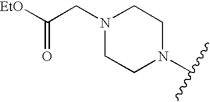 | 692/694 |
| 23 | 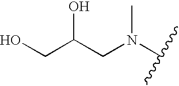 | 625/627 |
| 24 | 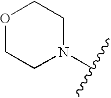 | 607/609 |
| 25 | 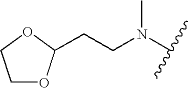 | 651/653 |
| 26 | 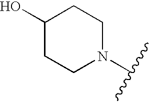 | 621/623 |
| 27 | 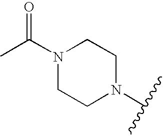 | 648/650 |

### Example 28

### 4-[3-Chloro-4-(1-ethyl-4,5-dimethyl-1H-imidazol-2-ylsulfanyl)-phenylamino]-6-methoxy-7-[3-(4-methyl-piperazin-1-yl)-propylamino]-quinoline-3-carbonitrile

### Step 1. 4-[3-Chloro-4-(1-ethyl-4,5-dimethyl-1H-imidazol-2-ylsulfanyl)-phenylamino]-7-fluoro-6-methoxy-quinoline-3-carbonitrile

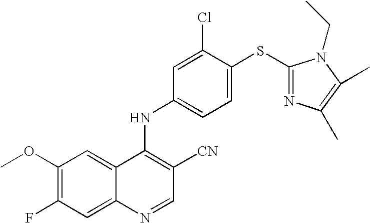

To a stirred solution of 4-chloro-7-fluoro-6-methoxy-quinoline-3-carbonitrile (0.504 g, 2.13 mmol) in ethoxyethanol (12 mL) was added 3-chloro-4-(1-ethyl-4,5-dimethyl-1H-imidazol-2-ylsulfanyl)-phenylamine (0.600 g, 2.13 mmol) and pyridine HCl (0.271 g, 2.34 mmol). The mixture was heated at 130° C., maintaining the temperature overnight. The solution was cooled to room temperature, then quenched with saturated NaHCO₃ solution (20 mL), stirred at room temperature for 30 minutes and the resultant precipitate was collected via vacuum filtration and then washed with water and diethyl ether to afford the product (0.882 g, 1.83 mmol, 85%) as a solid; ¹H NMR (DMSO-d₆) δ 1.03 (t, J=14.3 Hz, 3H), 2.06 (s, 3H), 2.13 (s, 3H), 3.85-3.92 (m, 5H), 6.52 (d, J=8.6 Hz, 1H), 6.81 (bs, 1H), 7.01 (s, 1H), 7.35 (d, J=12.2 Hz, 1H), 7.84 (d, J=9.6 Hz, 1H), 8.07 (s, 1H), 9.68 (s, 1H); mass spectrum [(+) ESI], m/z 482 (M+H).

### Step 2. 4-[3-Chloro-4-(1-ethyl-4,5-dimethyl-1H-imidazol-2-ylsulfanyl)-phenylamino]-6-methoxy-7-[3-(4-methyl-piperazin-1-yl)-propylamino]-quinoline-3-carbonitrile

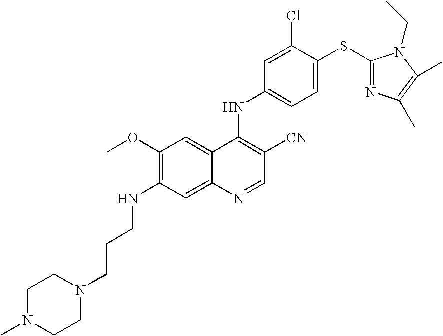

To a stirred solution of 4-[3-chloro-4-(1-ethyl-4,5-dimethyl-1H-imidazol-2-ylsulfanyl)-phenylamino]-7-fluoro-6-methoxy-quinoline-3-carbonitrile (0.45 g, 0.934 mmol) in NMP (15 mL) was added 1-(3-aminopropyl)-4-methylpiperazine (1.59 mL, 9.34 mmol). The mixture was heated at 115° C. overnight. The solution was cooled to room temperature, quenched with saturated NaHCO₃ solution (30 mL) and the precipitate was collected via vacuum filtration and washed with H₂O and Et₂O to afford the product (0.232 g, 3.75 mmol, 45%) as a solid; ¹H NMR (DMSO-d₆) δ 1.02 (t, J=14.4 Hz, 3H), 1.71-1.77 (m, 2H), 2.06 (s, 3H), 2.11 (s, 6H), 2.20-2.38 (m, 5H), 3.22-3.26 (m, 6H), 3.89-3.93 (m, 6H), 6.58 (d, J=8.7 Hz, 1H), 6.71-6.73 (m, 2H), 6.95 (dd, J=2.0, 8.6 Hz, 1H), 7.18 (d, J=2.0 Hz, 1H), 7.38 (s, 1H), 8.36 (s, 1H), 9.19 (s, 1H); mass spectrum [(+) ESI], m/z 619 (M+H).

### Example 29

### 4-[3-Chloro-4-(4,5-dimethyl-1-propyl-1H-imidazol-2-ylsulfanyl)-phenylamino]-7-(3-dimethylamino-propylamino)-6-methoxy-quinoline-3-carbonitrile

### Step 1. 4-[3-Chloro-4-(4,5-dimethyl-1-propyl-1H-imidazol-2-ylsulfanyl)-phenylamino]-7-fluoro-6-methoxy-quinoline-3-carbonitrile

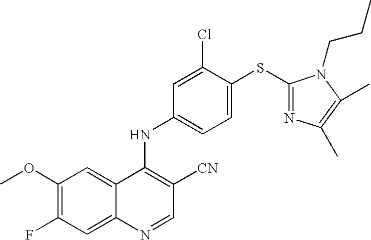

To a stirred solution of 4-chloro-7-fluoro-6-methoxy-quinoline-3-carbonitrile (0.0478 g, 0.202 mmol) in ethoxyethanol (2 mL) was added 3-chloro-4-(4,5-dimethyl-1-propyl-1H-imidazol-2-ylsulfanyl)-phenylamine (0.0478 g, 0.202 mmol) and pyridine HCl (0.0257 g, 0.222 mmol). The mixture was heated at 130° C. 2 hours. The solution was cooled to room temperature, then quenched with saturated NaHCO₃ solution (5 mL), stirred at room temperature for 30 minutes and the precipitate was collected via vacuum filtration to afford the product (0.067 g, 0.135 mmol, 67%) as a solid; ¹H NMR (DMSO-d₆) δ 0.77 (t, J=14.8 Hz, 3H), 1.45-1.49 (m, 2H), 2.07 (s, 3H), 2.13 (s, 3H), 3.80-3.85 (m, 5H), 6.56 (d, J=8.5 Hz, 1H), 6.71-6.73 (m, 1H), 6.87-6.92 (m, 1H), 7.67 (d, J=8.9 Hz, 1H), 7.84 (d, J=9.8 Hz, 1H), 8.05 (d, J=11.8 Hz, 1H), 9.08 (s, 1H); mass spectrum [(+) ESI], m/z 496 (M+H).

### Step 2. 4-[3-Chloro-4-(4,5-dimethyl-1-propyl-1H-imidazol-2-ylsulfanyl)-phenylamino]-7-(3-dimethylamino-propylamino)-6-methoxy-quinoline-3-carbonitrile

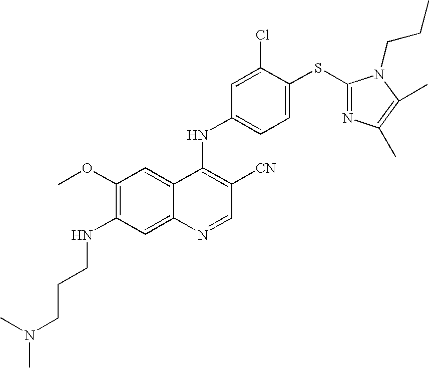

To a stirred solution of 4-[3-Chloro-4-(4,5-dimethyl-1-propyl-1H-imidazol-2-ylsulfanyl)-phenylamino]-7-fluoro-6-methoxy-quinoline-3-carbonitrile (0.067 g, 0.135 mmol) in NMP (2 mL) was added 3-dimethylamino-propylamine (0.17 mL, 1.35 mmol). The mixture was heated at 115° C. overnight. The solution was cooled to room temperature, quenched with water (5 mL), extracted with EtOAc (2×10 mL), dried over MgSO₄, filtered and then evaporated. The residue was purified by preparative TLC (15% MeOH: CHCl₃ gradient) to afford the product (0.010 g, 0.0173 mmol, 13%) as a solid; ¹H NMR (DMSO-d₆) δ 0.73 (t, J=14.8 Hz, 3H), 1.44-1.48 (m, 2H), 1.69-1.73 (m, 2H), 2.05 (s, 3H), 2.12 (s, 9H), 2.26-2.30 (m, 2H), 2.62-2.65 (m, 2H), 3.87 (t, J=14.9 Hz, 2H), 3.90 (s, 3H), 6.41-6.44 (m, 1H), 6.75 (s, 1H), 6.95 (dd, J=2.0, 8.6 Hz, 1H), 7.18 (d, J=2.0 Hz, 1H), 7.38 (s, 1H), 8.36 (s, 1H), 9.19 (s, 1H); mass spectrum [(+) ESI], m/z 578 (M+H).

### Example 30

### 4-[3-Chloro-4-(1-isopropyl-4,5-dimethyl-1H-imidazol-2-ylsulfanyl)-phenylamino]-7-(3-dimethylamino-propylamino)-6-methoxy-quinoline-3-carbonitrile

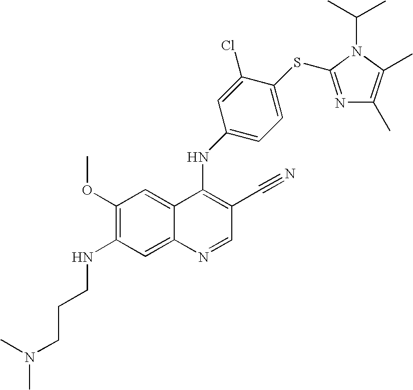

The compound was prepared as a solid (0.015 g, 15% for last step) from 3-chloro-4-(1-isopropyl-4,5-dimethyl-1H-imidazol-2-ylsulfanyl)-phenylamine in using a procedure similar to steps 1-2 of Example 29; ¹H NMR (DMSO-d₆) δ 1.32 (d, J=7.1 Hz, 6H), 1.69 (t, J=13.8 Hz, 2H), 2.04 (s, 3H), 2.12 (s, 6H), 2.21 (s, 3H), 2.27-2.29 (m, 4H), 3.87 (s, 3H), 4.64-4.70 (m, 1H), 6.41 (t, J=11.4 Hz, 1H), 6.53 (d, J=8.7 Hz, 1H), 6.75 (s, 1H), 6.95 (dd, J=2.0, 8.7 Hz, 1H), 7.18 (d, J=2.0 Hz, 1H), 7.38 (s, 1H), 8.36 (s, 1H), 9.19 (s, 1H); mass spectrum [(+) ESI], m/z 578 (M+H).

### Pharmacological Testing

IC₅₀ analysis was performed for selected compounds that were tested as inhibitors of IGF1-receptor kinase activity.

Biological testing was performed using a 96-well FRET (Lance) tyrosine kinase assay.

Materials:

Purified GST-IGFR; purified Bis-IGFR and Tris-IGFR; expressed in ExpresSF insect cells. Bis and Tris IGFR are made by phosphorylating the IGFR in the presence of ATP and Mg2+, followed by thrombin cleavage to remove the GST-thrombin cleavage site, and purification by HPLC. The Bis and tris IGFR are phosphorylated on two (bis) or all three tyrosines (tris) in the activation loop.
Biotinylated peptide with a sequence corresponding to the sequence surrounding the autophosphorylation sites in IGF1-R Biotin-NH₂-TRDIYETDYYRK-OH (Anaspec).
Europium-conjugated phosphotyrosine antibody (PT66) (Perkin-Elmer).

### Surelight APC (Perkin-elmer).

Plates: 96-well Assay plates (polypropylene plates, natural color).
96-well-Lance plates (Microfluor 2 black plates; Cat # 7205; Thermolabsystems.
ATP: (Amersham Pharmacia Cat#27-2056-01), 100 mM stock.
IGFR Lance Protocol (96-well format).

Procedure:

### Reagents:

Kinase Buffer (10×):0.5 M Hepes, pH 7.5 room temperature, 0.1 M MgCl₂.
Quench Buffer (to be added straight, 1:1, to r×n mix): 50 mM Hepes, pH 7.5 room temperature, 50 mM EDTA.
Lance Detection Buffer: 20 mM Tris-HCl, pH 7.5 room temperature, 0.15 M NaCl, 150 ug/ml BSA (Sigma Cat# A-7284).
Eu-labeled phosphotyrosine antibody PT66 (Perkin-Elmer): 1:4000 dilution of 200 μg/ml stock.
Streptavidin-APC (Perkin Elmer): 1/250 (4 microgram/ml final).
ATP (Amersham Pharmacia cat #27-2056-01) 100 mM stock: Diluted to 1 mM (10×) in water. 100 micromolar, final concentration in reaction.
Enzyme: GST-IGFR-12.5 ng/r×n (50 microliter 96 well assay); 5 ng/r×n (20 microliter 384 well assay); Bis-IGFR: 9 ng per 50 μL r×n; Tris-IGFR: 0.3 ng per 50 ul rxn.
Peptide: Anaspec; Biotin-NH₂-TRDIYETDYYRK-OH or Anaspec 610SAXAB, 2 micromolar final concentration.
Reaction: Prepared a mix containing the following relative amounts of reaction components (Include a no ATP control for Bkg subtraction):5λ 10× kinase buffer, 0.5% 100× peptide, 5λ 1.5 mg/ml BSA (Sigma # A-8918 BSA), 0.5×100% DMSO—DMSO is adjusted to a final conc of 1%, 32λ water. Mixed, then added IGFR followed by 5λ1 mM ATP. Allowed reaction to proceed for 1 hour, then added 50λ of quench buffer. To a separate plate added 50λ well of Lance detection buffer with PT66 and APC-streptavidin. Transferred 12.5λ of the kinase reaction to the antibody detection plate. Allowed antibody to react for 1 hour at room temperature in the dark. Measured in Victor with an excitation filter of 340 nm and an emission filter of 665 nm.

Compound additions: Selected compounds for testing were added from a 10% DMSO/50 mM Hepes (pH 7.5) 10× stock. 5 microliters of compound in this buffer are added to the enzyme-peptide-buffer mix. After 10 minutes incubation at room temperature, ATP is added to start the reaction. A no ATP background control is included in the assay.

Analysis of Results —IC₅₀ data. Absorbence data was read off of the Victor and was converted to a percentage of the control (untreated) values. IC₅₀ values were graphed and the point at which a 50% inhibition of the control level was determined. A hyperbolic model that forces a curve between 0% inhibition and 100% inhibition was used. If the model was not able to determine an IC₅₀, the IC₅₀ was assigned to the lowest concentration that gives a 50% or greater decrease in absorbence compared to control levels. If the calculated IC₅₀ value for a cell line exceeds the top concentration for the assay (typically 50 μg/mL), the assay was scored as >50.

IC₅₀ data and selectivity ratios of selected compounds are summarized in Table 5.

TABLE 5
IC₅₀ data and selectivity ratios of selected compounds

| Example | IGFR IC₅₀ (μM) |
|---|---|
| 1 | 0.031 |
| 2 | 0.035 |
| 3 | 0.291 |
| 4 | 0.12 |
| 5 | 0.03 |
| 6 | 0.394 |
| 7 | 17.2 |
| 8 | 0.013 |
| 9 | 0.021 |
| 10 | 3.88 |
| 11 | 0.024 |
| 12 | 0.03 |
| 13 | 0.041 |
| 14 | 0.027 |
| 15 | 0.142 |
| 16 | 0.035 |
| 17 | 0.005 |
| 18 | 0.011 |
| 19 | 0.014 |
| 20 | 0.02 |
| 21 | 0.067 |
| 22 | 0.017 |
| 23 | 0.014 |
| 24 | 0.022 |
| 25 | 0.013 |
| 26 | 0.047 |
| 27 | 0.016 |
| 28 | 0.002 |
| 29 | 0.161 |
| 30 | 0.071 |

What is claimed is:

1. A compound of formula I: 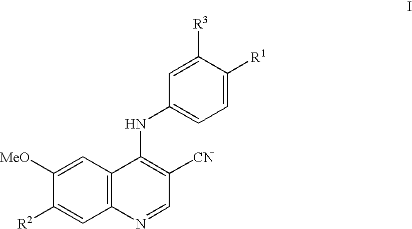
and pharmaceutically acceptable salts thereof;
wherein
R¹ is 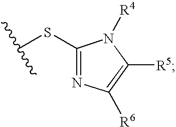
R² is —XCH₂CH₂CH₂Y;
R³ is selected from H, F, Cl, Br and I;
R⁴, R⁵ and R⁶ are each independently selected from unbranched alkyl of 1-6 carbon atoms, branched alkyl of 3-8 carbon atoms, aryl and benzyl, or R⁵ and R⁶ join together to form a fused 6-membered aryl ring comprising CR⁷ or heteroaryl ring comprising CR⁷ and N;
R⁷ and R⁸ are each independently selected from H, unbranched alkyl group of 1-6 carbon atoms, branched alkyl of 3-8 carbon atoms, hydroxyalkyl of 1-6 carbon atoms, dihydroxyalkyl of 1-6 carbon atoms, alkylsulfonyl of 1-6 carbon atoms, aryl sulfonyl, and —CH₂CH₂-heterocycle;
X is —O—, —N(H)—, or —N(Me)—; and
Y is selected from NR⁷R⁸, aryl, and a 5- to 6-membered heterocycle comprising 1-2 heteroatoms selected from N and O, said aryl and heterocycle substituted with 0-3 substituents selected from unbranched alkyl of 1-6 carbon atoms, branched alkyl of 3-8 carbon atoms, hydroxyalkyl of 1-6 carbon atoms, —OH, acetyl, —CO₂-alkyl of 1-6 carbon atoms, and —CH₂CO₂-alkyl of 1-6 carbon atoms.

2. The compound of claim 1, wherein R¹ is a di-substituted imidazole ring.

3. The compound of claim 1, wherein R¹ is a tri-substituted imidazole ring.

4. The compound of claim 3, wherein at least two of R⁴, R⁵ and R⁶ are each independently selected from unbranched alkyl of 1-6 carbon atoms and branched alkyl of 3 to 8 atoms.

5. The compound of claim 3, wherein R⁴, R⁵ and R⁶ are each independently selected from unbranched alkyl of 1-6 carbon atoms and branched alkyl of 3 to 8 atoms.

6. The compound of claim 3, wherein each of R⁵ and R⁶ are methyl.

7. The compound of claim 6, wherein R⁴ is ethyl or isopropyl.

8. The compound of claim 7, wherein X is —O— and Y is a 6-membered heterocycle comprising 1-2 heteroatoms selected from N and O.

9. The compound of claim 7, wherein X is —O— and Y is morpholine or piperazine, where Y is substituted with alkyl of 1-6 carbon atoms.

10. The compound of claim 9, wherein X is —O— and Y is 4-ethyl piperazine or 4-methyl piperazine.

11. A compound selected from: 4-({3-Chloro-4-[(1,4,5-trimethyl-1H-imidazol-2-yl)thio]phenyl}amino-7-[3-(dimethylamino)propyl]amino-6-methoxyquinoline-3-carbonitrile, 4-({4-[(1-benzyl-4,5-dimethyl-1H-imidazol-2-yl)thio]-3-chloro-phenyl}amino)-7-{[3-(dimethylamino)propyl]amino}-6-methoxyquinoline-3-carbonitrile, 4-({3-chloro-4-[(1,5-dimethyl-1H-benzimidazol-2-yl)thio]phenyl}amino)-7-{[3-(dimethylamino)propyl]amino}-6-methoxyquinoline-3-carbonitrile, 4-({3-chloro-4-[(1-ethyl-4,5-dimethyl-1H-imidazol-2-yl)thio]phenyl}amino)-7-{[3(dimethylamino)-propyl]amino}-6-methoxyquinoline-3-carbonitrile, 4-({3-bromo-4-[(1,4,5-trimethyl-1H-imidazol-2-yl)thio]phenyl}amino)-7-{[3-(dimethylamino)-propyl]amino}-6-methoxy-quinoline-3-carbonitrile, 7-{[3-(dimethyl-amino)propyl]-amino}-6-methoxy-4-({4-[(1,4,5-trimethyl-1 Himidazol-2-yl)thio]phenyl}amino)-quinoline-3-carbonitrile, 4-({3-Chloro-4-[(1-ethyl-4,5-dimethyl-1H-imidazol-2-yl)thio]phenyl}-amino)-6-methoxy-7-[(3-phenylpropyl)amino]quinoline-3-carbonitrile, 4-({3-chloro-4-[(1-ethyl-4,5-dimethyl-1H-imidazol-2-yl)thio]phenyl}-amino)-6-methoxy-7-[(3-morpholin-4-ylpropyl)amino]-quinoline-3-carbonitrile, 7-({3-[bis(2-hydroxyethyl)amino]propyl}amino)-4-({3-chloro-4-[(1-ethyl-4,5-dimethyl-1H-imidazol-2-yl)thio]phenyl}amino)-6-methoxyquinoline-3-carbonitrile, N-(3-{4-[3-chloro-4-(1-ethyl-4,5-dimethyl-1H-imidazol-2-ylsulfanyl)-phenylamino]-3-cyano-6-methoxy-quinolin-7-yloxy}-propyl)-benzenesulfonamide, 7-{3-[tert-butyl(2-hydroxy-ethyl)amino]propoxy}-4-({3-chloro-4-[(1-ethyl-4,5-dimethyl-1H-imidazol-2-yl)thio]phenylamino)-6-methoxyquinoline-3-carbonitrile, 4-({3-chloro-4-[(1-ethyl-4,5-dimethyl-1H-imidazol-2-yl)thio]phenyl}amino)-7-[3-(dimethyl-amino)propoxy]-6-methoxyquinoline-3-carbonitrile, ethyl 4-(3-{[4-({3-chloro-4-[(1-ethyl-4,5-dimethyl-1H-imidazol-2-yl)thio]phenyl}amino)-3-cyano-6-methoxyquinolin-7-yl]oxy}propyl)-piperazine-1-carboxylate, 4-({3-chloro-4-[(1-ethyl-4,5-dimethyl-1H-imidazol-2-yl)thio]phenyl}amino)-7-[3-(2-ethylpiperidin-1-yl)propoxy]-6-methoxy-quinoline-3-carbonitrile, ethyl 1-(3-{[4-({3-chloro-4-[(1-ethyl-4,5-dimethyl-1H-imidazol-2-yl)thio]phenyl}amino)-3-cyano-6-methoxyquinolin-7-yl]oxy}propyl)-piperidine-4-carboxylate, N-(3-{[4-({3-chloro-4-[(1-ethyl-4,5-dimethyl-1H-imidazol-2-yl)thio]phenyl}amino)-3-cyano-6-methoxyquinolin-7-yl]oxy}propyl)-methanesulfonamide, 4-({3-Chloro-4-[(1-ethyl-4,5-dimethyl-1H-imidazol-2-yl)sulfanyl]phenyl}amino)-7-[3-(4-ethyl-1-piperazinyl)-propoxy]-6-methoxy-3-quinolinecarbonitrile, 4-({3-chloro-4-[(1-ethyl-4,5-dimethyl-1H-imidazol-2-yl)thio]-phenyl}amino)-7-{3-[4-(2-hydroxyethyl)piperazin-1-yl]propoxy}-6-methoxy-quinoline-3-carbonitrile, 4-({3-chloro-4-[(1-ethyl-4,5-dimethyl-1H-imidazol-2-yl)thio]phenyl}amino)-7-{3-[(2-hydroxyethyl)(methyl)amino]propoxy}-6-methoxy-quinoline-3-carbonitrile, 4-({3-chloro-4-[(1-ethyl-4,5-dimethyl-1H-imidazol-2-yl)thio]phenyl}amino)-7-[3-(3-hydroxypyrrolidin-1-yl)propoxy]-6-methoxyquinoline-3-carbonitrile, 4-({3-chloro-4-[(1-ethyl-4,5-dimethyl-1H-imidazol-2-yl)thio]phenyl}amino)-6-methoxy-7-(3-pyrrolidin-1-ylpropoxy)quinoline-3-carbonitrile, ethyl [4-(3-{[4-({3-chloro-4-[(1-ethyl-4,5-dimethyl-1H-imidazol-2-yl)thio]phenyl}amino)-3-cyano-6-methoxyquinolin-7-yl]oxy}propyl)piperazin-1-yl]acetate, 4-({3-chloro-4-[(1-ethyl-4,5-dimethyl-1H-imidazol-2-yl)thio]phenyl}amino)-7-{3-[(2,3-dihydroxypropyl)(methyl)-amino]propoxy}-6-methoxyquinoline-3-carbonitrile, 4-({3-chloro-4-[(1-ethyl-4,5-dimethyl-1H-imidazol-2-yl)thio]phenyl}amino)-6-methoxy-7-(3-morpholin-4-ylpropoxy)qquinoline-3-carbonitrile, 4-({3-chloro-4-[(1-ethyl-4,5-dimethyl-1H-imidazol-2-yl)thio]phenyl}amino)-7-{3-[[2-(1,3-dioxolan-2-yl)ethyl](methyl)amino]propoxy}-6-methoxyquinoline-3-carbonitrile, 4-({3-chloro-4-[(1-ethyl-4,5-dimethyl-1H-imidazol-2-yl)thio]phenyl}amino)-7-[3-(4-hydroxypiperidin-1-yl)propoxy]-6-methoxyquinoline-3-carbonitrile, 7-[3-(4-acetylpiperazin-1-yl)propoxy]-4-({3-chloro-4-[(1-ethyl-4,5-dimethyl-1H imidazol-2-yl)thio]phenyl}amino)-6-methoxyquinoline-3-carbonitrile, 4-[3-Chloro-4-(1-ethyl-4,5-dimethyl-1H-imidazol-2-ylsulfanyl)-phenylamino]-6-methoxy-7-[3-(4-methyl-piperazin-1-yl)-propylamino]-quinoline-3-carbonitrile, 4-[3-Chloro-4-(4,5-dimethyl-1-propyl-1H-imidazol-2-ylsulfanyl)-phenylamino]-7-(3-dimethylamino-propylamino)-6-methoxy-quinoline-3-carbonitrile, 4-[3-Chloro-4-(1-isopropyl-4,5-dimethyl-1H-imidazol-2-ylsulfanyl)-phenylamino]-7-(3-dimethylamino-propylamino)-6-methoxy-quinoline-3-carbonitrile and pharmaceutically acceptable salts thereof.

12. A method for making a compound of claim 1, comprising the step of:
reacting a compound of formula 1: 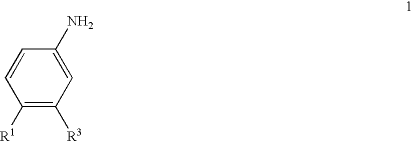
with a compound of formula 2:

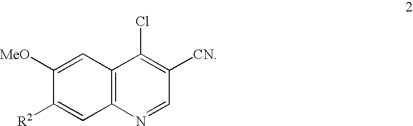

13. The method of claim 12, wherein R² is F.

14. The method of claim 13, wherein the compound of formula 1 and the compound of formula 2 are reacted in the presence of pyridine hydrochloride and heated at 130° C. for 2 hours.

15. A pharmaceutical composition comprising a compound according to any of claims 1-11 and a pharmaceutically acceptable carrier.

16. A pharmaceutical composition comprising a compound according to any of claims 1-11 in combination with one or more other kinase-inhibiting pharmaceutical compositions or chemotherapeutic agents, and a pharmaceutically acceptable carrier.

17. The pharmaceutical composition of claims 15 or 16, capable of inhibiting an insulin growth factor receptor kinase.

18. A method of treating a disease associated with inhibiting insulin growth factor receptor kinase activity in a mammal comprising administering to the mammal a kinase-inhibiting amount of a compound according to any one of claims 1-11.

19. The method of claim 18, wherein the disease is associated with an insulin growth factor receptor kinase dependent condition.

20. The method of claim 19 wherein the disease comprises inflammation or cancer.

21. The method of claim 20, wherein the cancer is selected from the group consisting of: breast, kidney, bladder, thyroid, mouth, larynx, esophagus, stomach, colon, ovary, lung, pancreas, skin, liver, prostate and brain cancer.
